(19) United States
(12) Patent Application Publication
(10) Pub. No.: US 2023/0382941 A1
(43) Pub. Date: Nov. 30, 2023

(54) SPHERICAL NUCLEIC ACIDS WITH DENDRITIC LIGANDS

(71) Applicant: NORTHWESTERN UNIVERSITY, Evanston, IL (US)

(72) Inventors: Chad A. Mirkin, Wilmette, IL (US); Katherine E. Bujold, Magog (CA); Max Everett Distler, Evanston, IL (US)

(21) Appl. No.: 18/225,400

(22) Filed: Jul. 24, 2023

Related U.S. Application Data
(63) Continuation of application No. 17/076,607, filed on Oct. 21, 2020.
(60) Provisional application No. 62/923,923, filed on Oct. 21, 2019.

(51) Int. Cl. C07H 19/04; C07K 14/705; C12N 15/117; C07K 16/00

(52) U.S. Cl. CPC C07H 19/04; C07K 14/705; C12N 15/117; C07K 16/00

(57) ABSTRACT

The present disclosure is directed to spherical nucleic acids (SNAs) comprising a nanoparticle core and an oligonucleotide dendron attached thereto. The disclosure also provides methods of using the SNAs for, for example, gene regulation and immune regulation.

### CROSS-REFERENCE TO RELATED APPLICATIONS

This application is a Continuation of U.S. application Ser. No. 17/076,607, filed Oct. 21, 2020, which claims the priority benefit under 35 U.S.C. § 119(e) of U.S. Provisional Patent Application No. 62/923,923, filed Oct. 21, 2019, each of which is incorporated herein by reference in its entirety.

### STATEMENT OF GOVERNMENT INTEREST

This invention was made with government support under grant number N00014-15-1-0043 awarded by the Office of Naval Research and grant number FA8650-15-2-5518 awarded by the Air Force Research Lab. The government has certain rights in the invention.

### BACKGROUND

Cellular delivery of nanoscale materials and biomolecules is consequential, as their catalytic and structural properties impact cellular behavior and viability. However, their low cellular uptake, due to the hydrophilicity and size, greatly limits their translation as therapeutics. DNA ligands have been shown to increase the cellular delivery of many nanoscale materials including proteins. However, this property typically requires a dense shell of DNA to be achieved, effectively limiting its applicability to large, stable biomolecules.

### SUMMARY

The present disclosure is generally related to the fields of Spherical Nucleic Acids (SNAs) and Programmable Atom Equivalents (PAEs). More specifically, the disclosure relates to dendrimeric spherical nucleic acids and dendrimeric programmable atom equivalents. The dendrimer and dendron strategies provided herein reduce the required number of available attachment points for efficient SNA and PAE functionalization. The strategies provided herein also enable cellular uptake without jeopardizing protein structure and function.

Applications of the technology disclosed herein include, but are not limited to,
SNA-Based Therapeutics
PAEs
Nanomaterials Delivery
Protein Therapeutics

Advantages of the technology disclosed herein include, but are not limited to,
Functionalization of proteins, liposomes, gold clusters, inorganic nanoparticle cores, micelles, polymers
Efficient functionalization of nanoparticle cores with few accessible sites
Enhancement of cellular uptake
Well defined and monodisperse SNAs and PAEs for crystallization

Methods of the disclosure include but are not limited to the synthesis of nucleic acid (e.g., DNA) dendrons (and, in some embodiments, dendrimers) and subsequent conjugation to nanoparticle cores, yielding densely functionalized PAEs and SNAs that can participate in assembly and cellular uptake respectively. Amino functionalized DNA dendrons synthesized using phosphoramidite chemistry, purified by PAGE or HPLC, and analyzed by PAGE and MALDI-TOF. Conjugation of DNA dendrons on proteins with available surface cysteines and/or lysines using small chemical linkers and subsequent purification using affinity columns or size exclusion. The dendron (and in some embodiments, dendrimer)-protein conjugates were characterized by SDS-PAGE and Analytical SEC. They show enhanced cellular uptake compared to native proteins by FACS and enzymatic activity is not affected. These properties can be expanded to other nanoparticle cores using thiol- and azide-modified DNA dendrons.

The strategies provided herein allow one to use a minimal number of conjugation sites to maximize nucleic acid (e.g., DNA) loading and cellular uptake/assembly, without jeopardizing nanoparticle core function.

Accordingly, in some aspects the disclosure provides a spherical nucleic acid (SNA) comprising a nanoparticle core and an oligonucleotide dendron attached to the external surface of the nanoparticle core, wherein the oligonucleotide dendron comprises an oligonucleotide stem linked to a plurality of oligonucleotide branches through one or more of a doubler moiety, a trebler moiety, or a combination thereof. In some embodiments, the oligonucleotide dendron is a DNA dendron, a RNA dendron, a modified oligonucleotide dendron, or a combination thereof.

In some embodiments, the oligonucleotide dendron comprises about 2 to about 27 oligonucleotide branches. In some embodiments, the oligonucleotide dendron comprises 6 oligonucleotide branches. In further embodiments, the oligonucleotide dendron comprises 9 oligonucleotide branches. In some embodiments, the doubler moiety comprises a structure prior to incorporation into the oligonucleotide dendron that is:

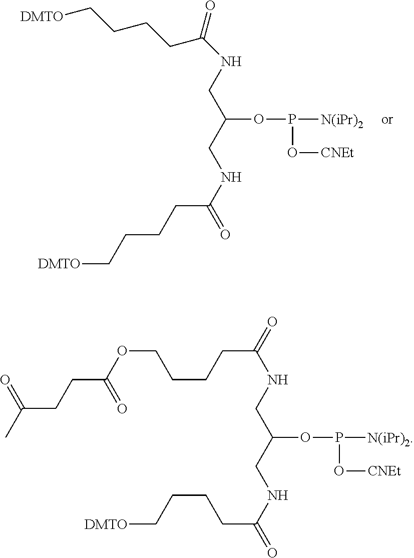

DMT=4,4′-dimethoxytriryl; O=oxygen; C=carbon; N=nitrogen; Et=ethyl; iPr=isopropyl. In some embodiments, the trebler moiety comprises a structure prior to incorporation into the oligonucleotide dendron that is:

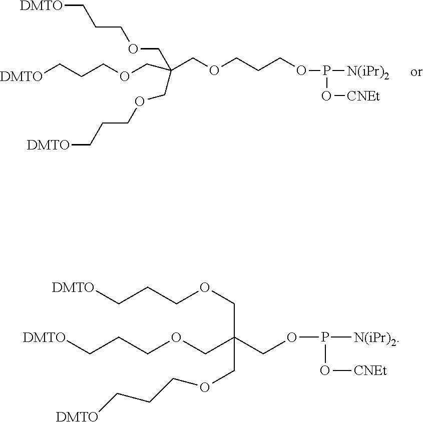

4,4′-dimethoxytriryl; O=oxygen; C=carbon; N=nitrogen; Et=ethyl; iPr=isopropyl. In some embodiments, the oligonucleotide stem comprises an inhibitory oligonucleotide. In some embodiments, one or more of the plurality of oligonucleotide branches comprises an inhibitory oligonucleotide. In further embodiments, the inhibitory oligonucleotide is antisense DNA, small interfering RNA (siRNA), an aptamer, a short hairpin RNA (shRNA), a DNAzyme, or an aptazyme. In some embodiments, the oligonucleotide stem comprises an immunostimulatory oligonucleotide. In some embodiments, one or more of the plurality of oligonucleotide branches comprises an immunostimulatory oligonucleotide. In further embodiments, the immunostimulatory oligonucleotide is a toll-like receptor (TLR) agonist. In some embodiments, the TLR agonist is a toll-like receptor 1 (TLR-1) agonist, toll-like receptor 2 (TLR-2) agonist, toll-like receptor 3 (TLR-3) agonist, toll-like receptor 4 (TLR-4) agonist, toll-like receptor 5 (TLR-5) agonist, toll-like receptor 6 (TLR-6) agonist, toll-like receptor 7 (TLR-7) agonist, toll-like receptor 8 (TLR-8) agonist, toll-like receptor 9 (TLR-9) agonist, toll-like receptor 10 (TLR-10) agonist, toll-like receptor 11 (TLR-11) agonist, toll-like receptor 12 (TLR-12) agonist, toll-like receptor 13 (TLR-13) agonist, or a combination thereof. In some embodiments, the oligonucleotide stem comprises a toll-like receptor (TLR) antagonist. In further embodiments, one or more of the plurality of oligonucleotide branches comprises a toll-like receptor (TLR) antagonist. In still further embodiments, the TLR-antagonist is a toll-like receptor 1 (TLR-1) antagonist, toll-like receptor 2 (TLR-2) antagonist, toll-like receptor 3 (TLR-3) antagonist, toll-like receptor 4 (TLR-4) antagonist, toll-like receptor 5 (TLR-5) antagonist, toll-like receptor 6 (TLR-6) antagonist, toll-like receptor 7 (TLR-7) antagonist, toll-like receptor 8 (TLR-8) antagonist, toll-like receptor 9 (TLR-9) antagonist, toll-like receptor 10 (TLR-10) antagonist, toll-like receptor 11 (TLR-11) antagonist, toll-like receptor 12 (TLR-12) antagonist, toll-like receptor 13 (TLR-13) antagonist, or a combination thereof. In some embodiments, the oligonucleotide stem comprises an additional agent. In some embodiments, one or more of the plurality of oligonucleotide branches comprises an additional agent. In further embodiments, the additional agent is a protein, a small molecule, a peptide, or a combination thereof. In some embodiments, the protein is an antibody. In some embodiments, the oligonucleotide stem of the oligonucleotide dendron is attached to the nanoparticle core through a thiol linkage, a lipid anchor group, or is attached to a lysine or cysteine residue of the nanoparticle core. In further embodiments, the lipid anchor group is a cholesterol or tocopherol. In some embodiments, an SNA of the disclosure further comprises one or more additional oligonucleotide dendrons, wherein each of the one or more additional oligonucleotide dendrons comprises a plurality of oligonucleotide branches linked by a doubler moiety, a trebler moiety, or a combination thereof. In some embodiments, each of the one or more additional oligonucleotide dendrons comprises about 2 to about 27 oligonucleotide branches. In some embodiments, each of the one or more additional oligonucleotide dendrons comprises 6 oligonucleotide branches. In further embodiments, each of the one or more additional oligonucleotide dendrons comprises 9 oligonucleotide branches. In some embodiments, each oligonucleotide dendron attached to the SNA comprises the same number of oligonucleotide branches. In some embodiments, at least two of the oligonucleotide dendrons attached to the SNA comprises a different number of oligonucleotide branches relative to each other. In some embodiments, one or more of the oligonucleotide stems of the one or more additional oligonucleotide dendrons comprises an inhibitory oligonucleotide. In some embodiments, one or more of the plurality of oligonucleotide branches of the one or more additional oligonucleotide dendrons comprises an inhibitory oligonucleotide. In further embodiments, the inhibitory oligonucleotide is antisense DNA, small interfering RNA (siRNA), an aptamer, a short hairpin RNA (shRNA), a DNAzyme, or an aptazyme. In some embodiments, one or more of the oligonucleotide stems of the one or more additional oligonucleotide dendrons comprises an immunostimulatory oligonucleotide. In some embodiments, one or more of the plurality of oligonucleotide branches of the one or more additional oligonucleotide dendrons comprises an immunostimulatory oligonucleotide. In further embodiments, the immunostimulatory oligonucleotide is a toll-like receptor (TLR) agonist. In various embodiments, the TLR agonist is a toll-like receptor 1 (TLR-1) agonist, toll-like receptor 2 (TLR-2) agonist, toll-like receptor 3 (TLR-3) agonist, toll-like receptor 4 (TLR-4) agonist, toll-like receptor 5 (TLR-5) agonist, toll-like receptor 6 (TLR-6) agonist, toll-like receptor 7 (TLR-7) agonist, toll-like receptor 8 (TLR-8) agonist, toll-like receptor 9 (TLR-9) agonist, toll-like receptor 10 (TLR-10) agonist, toll-like receptor 11 (TLR-11) agonist, toll-like receptor 12 (TLR-12) agonist, toll-like receptor 13 (TLR-13) agonist, or a combination thereof. In some embodiments, one or more of the oligonucleotide stems of the one or more additional oligonucleotide dendrons comprises a toll-like receptor (TLR) antagonist. In some embodiments, one or more of the plurality of oligonucleotide branches of the one or more additional oligonucleotide dendrons comprises a toll-like receptor (TLR) antagonist. In further embodiments, the TLR-antagonist is a toll-like receptor 1 (TLR-1) antagonist, toll-like receptor 2 (TLR-2) antagonist, toll-like receptor 3 (TLR-3) antagonist, toll-like receptor 4 (TLR-4) antagonist, toll-like receptor 5 (TLR-5) antagonist, toll-like receptor 6 (TLR-6) antagonist, toll-like receptor 7 (TLR-7) antagonist, toll-like receptor 8 (TLR-8) antagonist, toll-like receptor 9 (TLR-9) antagonist, toll-like receptor 10 (TLR-10) antagonist, toll-like receptor 11 (TLR-11) antagonist, toll-like receptor 12 (TLR-12) antagonist, toll-like receptor 13 (TLR-13) antagonist, or a combination thereof. In some embodiments, one or more of the oligonucleotide stems of the one or more additional oligonucleotide dendrons comprises an additional agent. In some embodiments, one or more of the plurality of oligonucleotide branches of the one or more additional oligonucleotide dendrons comprises an additional agent. In further embodiments, the additional agent is a protein, a small molecule, or a peptide. In some embodiments, the protein is an antibody. In some embodiments, an SNA of the disclosure comprises about 1 to about 100 oligonucleotide dendrons. In some embodiments, an SNA of the disclosure comprises about 1 to about 10 oligonucleotide dendrons. In some embodiments, each of the one or more additional oligonucleotide dendrons is a DNA dendron, a RNA dendron, a modified oligonucleotide dendron, or a combination thereof. In some embodiments, each of the one or more additional oligonucleotide dendrons is a DNA dendron. In some embodiments, each of the one or more additional oligonucleotide dendrons is a RNA dendron. In some embodiments, each of the one or more additional oligonucleotide dendrons is a modified oligonucleotide dendron. In further embodiments, the one or more additional oligonucleotide dendrons comprises a mixture of DNA dendrons, RNA dendrons, and modified oligonucleotide dendrons. In some embodiments, the nanoparticle core is a metallic core, a semiconductor core, an insulator core, an upconverting core, a micellar core, a dendrimer core, a liposomal core, a polymer core, a metal-organic framework core, a protein core, or a combination thereof. In further embodiments, the polymer is polylactide, a polylactide-polyglycolide copolymer, a polycaprolactone, a polyacrylate, alginate, albumin, silica, polypyrrole, polythiophene, polyaniline, polyethylenimine, poly(methyl methacrylate), poly(lactic-co-glycolic acid) (PLGA), or chitosan. In some embodiments, the nanoparticle core is gold, silver, platinum, aluminum, palladium, copper, cobalt, indium, cadmium selenide, iron oxide, fullerene, metal-organic framework, zinc sulfide, or nickel. In some embodiments, the nanoparticle core is a protein core. In further embodiments, the protein core is an enzyme, a therapeutic protein, a structural protein, a defensive protein, a storage protein, a transport protein, a hormone, a receptor protein, a motor protein, an immunogenic protein, or a fluorescent protein. In some embodiments, the oligonucleotide stem of the oligonucleotide dendron is attached to a lysine or cysteine residue of the protein core. In some embodiments, the one or more additional oligonucleotide dendrons is attached to one or more lysine or cysteine residues of the protein core.

In some aspects, the disclosure provides a composition comprising a plurality of spherical nucleic acids (SNAs) of the disclosure.

In some aspects, a method of inhibiting expression of a gene is provided comprising the step of hybridizing a polynucleotide encoding the gene product with a spherical nucleic acid (SNA) or composition of the disclosure, wherein hybridizing between the polynucleotide and the oligonucleotide stem and/or one or more of the plurality of oligonucleotide branches of the oligonucleotide dendron occurs over a length of the polynucleotide with a degree of complementarity sufficient to inhibit expression of the gene product. In some embodiments, the hybridizing is between the polynucleotide and the oligonucleotide stem of the oligonucleotide dendron. In some embodiments, the hybridizing is between the polynucleotide and one or more of the plurality of oligonucleotide branches of the oligonucleotide dendron. In some embodiments, expression of the gene product is inhibited in vivo. In some embodiments, expression of the gene product is inhibited in vitro.

In some aspects, the disclosure provides a method for up-regulating activity of a toll-like receptor (TLR), comprising contacting a cell having the toll-like receptor with the spherical nucleic acid (SNA) or composition of the disclosure. In some embodiments, the oligonucleotide stem of the oligonucleotide dendron is a TLR agonist. In some embodiments, one or more of the plurality of oligonucleotide branches of the oligonucleotide dendron is a TLR agonist. In some embodiments, the toll-like receptor is chosen from the group consisting of toll-like receptor 1, toll-like receptor 2, toll-like receptor 3, toll-like receptor 4, toll-like receptor 5, toll-like receptor 6, toll-like receptor 7, toll-like receptor 8, toll-like receptor 9, toll-like receptor 10, toll-like receptor 11, toll-like receptor 12, and toll-like receptor 13.

In some aspects, the disclosure provides a method for down-regulating activity of a toll-like receptor (TLR), comprising contacting a cell having the toll-like receptor with a spherical nucleic acid (SNA) or composition of the disclosure. In some embodiments, the oligonucleotide stem is a TLR antagonist. In some embodiments, one or more of the plurality of oligonucleotide branches of the oligonucleotide dendron is a TLR antagonist. In further embodiments, the toll-like receptor is chosen from the group consisting of toll-like receptor 1, toll-like receptor 2, toll-like receptor 3, toll-like receptor 4, toll-like receptor 5, toll-like receptor 6, toll-like receptor 7, toll-like receptor 8, toll-like receptor 9, toll-like receptor 10, toll-like receptor 11, toll-like receptor 12, and toll-like receptor 13. In some embodiments, the method is performed in vitro. In some embodiments, the method is performed in vivo.

### BRIEF DESCRIPTION OF THE DRAWINGS

FIG. 1 shows the characterization of DNA dendrons (BD) by polyacrylamide gel electrophoresis (PAGE) and Time of Flight Matrix-Assisted Laser Desorption/Ionization Mass Spectrometry (MALDI-TOF) analysis. (A) depicts the structure of example oligonucleotide dendrons of the disclosure. (B) shows results of MALDI-TOF analysis and (C) depicts results of PAGE analysis.

FIG. 2 shows results of experiments demonstrating that DNA dendrons were taken up by C166 endothelial cells. (A) shows the increase in the percentage of cells positive for DNA, and (B) shows the increase in the amount of DNA in each cell.

FIG. 3 shows results of experiments demonstrating that DNA dendrons are also taken up by Antigen Presenting Cells (APCs). The percentage of cells positive for DNA (A) and the amount of DNA in each cell (B) increased for all dendrons tested relative to linear DNA (T20). The results showed that the six- and nine-branched DNA dendrons outcompeted the three-branched DNA dendron and the linear DNA control (T20) over a large range of concentrations.

FIG. 4 shows that dendron-peptide conjugates are taken up by antigen presenting cells (APCs). (A) shows that the six-branched dendron-Ova conjugate was taken up significantly more than the other sample, and (B) shows that the delivered peptide remained functional and could bind to its specific cell surface receptor.

FIG. 5 shows that DNA dendrons could be used as universal tags for cellular delivery. (A) shows sodium dodecyl sulfate-polyacrylamide gel electrophoresis (SDS-PAGE) characterization of mNeonGreen (mNG), a model protein, conjugated to a single DNA dendron. (B) shows that more mNeonGreen was taken up over time relative to the naked protein when conjugated to the six branched dendron.

FIG. 6 shows DNA dendron-enabled delivery of a therapeutic protein, and shows that cellular uptake of the protein (adenosine deaminase (ADA)) conjugated to an oligonucleotide dendron was significantly increased relative to that of the naked protein.

FIG. 7 shows the characterization of oligonucleotide dendrons with three, six, and nine oligonucleotide branches.

FIG. 8 demonstrates that protein-oligonucleotide dendron SNAs were synthesized successfully.

FIG. 9 shows that the T-SNA was efficiently taken up by HeLa cells. “T-SNA”=trebler SNA, therefore it is the 3BD SNA or the 3 branched dendron conjugated to the protein.

### DETAILED DESCRIPTION

The present disclosure is directed to spherical nucleic acids (SNAs) comprising a nanoparticle core and an oligonucleotide dendron attached to the external surface of the nanoparticle core.

Spherical nucleic acids derive their properties from the dense packing of radially oriented oligonucleotides onto the surface of a nanoparticle core. While this strategy has found tremendous success in both therapeutic and materials-based applications, it is not yet easily amenable to all types of cores. Among them, proteins, liposomes, gold clusters, micelles, polymers and other inorganic nanoparticles have often suffered from poor SNA and PAE-like properties due to low packing of oligonucleotides on their surface. Provided herein is a dendron-based strategy that can effectively increase the number of oligonucleotides appended onto a given attachment site. Using proteins as a challenging model system due to their high sensitivity to denaturation, lack of functionalization sites and mild synthesis requirements, it is shown herein that the dendron strategy can be applied without significantly affecting protein structure and function. Moreover, these SNAs were synthesized using a versatile protocol that can be conducted under mild conditions and is easily amenable to a variety of cores. The resulting dendrimeric-protein conjugates exhibit increased cellular uptake compared to native proteins consistent with a successful SNA formation. Moreover, it is superior to typical proSNA functionalization strategies which involve the conjugation of a single oligonucleotide per attachment point. Taken together, this strategy emphasizes the importance of rational DNA design in improving the next generations of PAEs and SNAs.

As used in this specification and the appended claims, the singular forms “a,” “an,” and “the” include plural reference unless the context clearly dictates otherwise.

The terms “polynucleotide” and “oligonucleotide” are interchangeable as used herein.

A “dendron” as used herein refers to an individual oligonucleotide molecule comprising an oligonucleotide stem linked to a plurality of oligonucleotide branches through one or more of a doubler moiety, a trebler moiety, or a combination thereof. A “dendrimer” as used herein refers to a spherical or substantially spherical structure, whereby multiple dendrons are attached to and extend radially outward from a nanoparticle core.

A “linker” as used herein is a moiety that joins an oligonucleotide (e.g., an oligonucleotide stem) to a nanoparticle core (e.g., a protein core) of a spherical nucleic acid (SNA), as described herein. In any of the aspects or embodiments of the disclosure, a linker is a cleavable linker, a non-cleavable linker, a traceless linker, or a combination thereof. In various embodiments, thiol modifications on the dendron may be used for gold nanoparticle cores (cleavable). The crosslinkers succinimidyl 3-(2-pyridyldithio)propionate (cleavable), NHS-PEG4-Azide (non-cleavable), and 4-nitrophenyl 2-(2-pyridyldithio)ethyl carbonate (traceless) may be used for protein and peptide cores.

As used herein, an “oligonucleotide stem” is an oligonucleotide that is attached on one end to a nanoparticle core or a linker and on the other end to a doubler moiety, a trebler moiety, or a combination thereof.

As used herein, an “oligonucleotide branch” is an oligonucleotide that is connected to an oligonucleotide stem through one or more doubler moieties, trebler moieties, or a combination thereof.

As used herein, the term “about,” when used to modify a particular value or range, generally means within 20 percent, e.g., within 10 percent, 5 percent, 4 percent, 3 percent, 2 percent, or 1 percent of the stated value or range.

Unless otherwise stated, all ranges contemplated herein include both endpoints and all numbers between the endpoints. The use of “about” or “approximately” in connection with a range applies to both ends of the range. Thus, “about 20 to 30” is intended to cover “about 20 to about 30”, inclusive of at least the specified endpoints.

### Spherical Nucleic Acids (SNAs)

In any of the aspects or embodiments of the disclosure, a spherical nucleic acid (SNA) comprises a nanoparticle core and an oligonucleotide dendron attached thereto. In some embodiments, an SNA further comprises an oligonucleotide that is not an oligonucleotide dendron (also referred to herein as a non dendron oligonucleotide). In some embodiments, an SNA further comprises a plurality of oligonucleotides that are not oligonucleotide dendrons in addition to one or more oligonucleotide dendrons. In further embodiments, the plurality of oligonucleotides comprises from about 1 to about 50 oligonucleotides.

SNAs can range in size from about 1 nanometer (nm) to about 500 nm, about 1 nm to about 400 nm, about 1 nm to about 300 nm, about 1 nm to about 200 nm, about 1 nm to about 150 nm, about 1 nm to about 100 nm, about 1 nm to about 90 nm, about 1 nm to about 80 nm in diameter, about 1 nm to about 70 nm in diameter, about 1 nm to about 60 nm in diameter, about 1 nm to about 50 nm in diameter, about 1 nm to about 40 nm in diameter, about 1 nm to about 30 nm in diameter, about 1 nm to about 20 nm in diameter, about 1 nm to about 10 nm, about 10 nm to about 150 nm in diameter, about 10 nm to about 140 nm in diameter, about 10 nm to about 130 nm in diameter, about 10 nm to about 120 nm in diameter, about 10 nm to about 110 nm in diameter, about 10 nm to about 100 nm in diameter, about 10 nm to about 90 nm in diameter, about 10 nm to about 80 nm in diameter, about 10 nm to about 70 nm in diameter, about 10 nm to about 60 nm in diameter, about 10 nm to about 50 nm in diameter, about 10 nm to about 40 nm in diameter, about 10 nm to about 30 nm in diameter, or about 10 nm to about 20 nm in diameter. In further aspects, the disclosure provides a plurality of SNAs, each SNA comprising one or more oligonucleotide dendrons (and optionally one or more oligonucleotides that are not oligonucleotide dendrons) attached thereto. Thus, in some embodiments, the size of the plurality of SNAs is from about 10 nm to about 150 nm (mean diameter), about 10 nm to about 140 nm in mean diameter, about 10 nm to about 130 nm in mean diameter, about 10 nm to about 120 nm in mean diameter, about 10 nm to about 110 nm in mean diameter, about 10 nm to about 100 nm in mean diameter, about 10 nm to about 90 nm in mean diameter, about 10 nm to about 80 nm in mean diameter, about 10 nm to about 70 nm in mean diameter, about 10 nm to about 60 nm in mean diameter, about 10 nm to about 50 nm in mean diameter, about 10 nm to about 40 nm in mean diameter, about 10 nm to about 30 nm in mean diameter, or about 10 nm to about 20 nm in mean diameter. In some embodiments, the diameter (or mean diameter for a plurality of SNAs) of the SNAs is from about 10 nm to about 150 nm, from about 30 to about 100 nm, or from about 40 to about 80 nm. In some embodiments, the size of the nanoparticles used in a method varies as required by their particular use or application. The variation of size is advantageously used to optimize certain physical characteristics of the SNAs, for example, the amount of surface area to which oligonucleotides may be attached as described herein. It will be understood that the foregoing diameters of SNAs can apply to the diameter of the nanoparticle core itself or to the diameter of the nanoparticle core and the one or more oligonucleotide dendrons attached thereto.

### Oligonucleotides

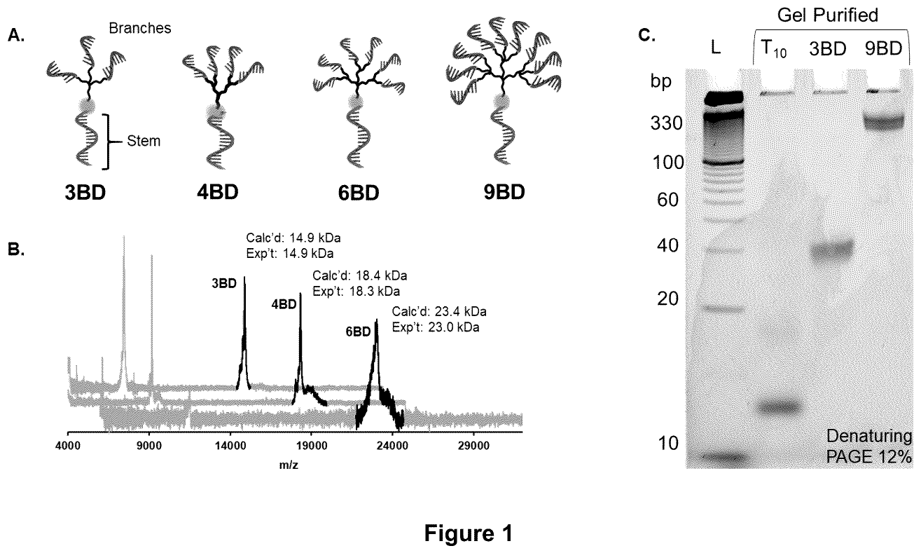

The disclosure provides spherical nucleic acids (SNAs) comprising a nanoparticle core and an oligonucleotide dendron attached to the external surface of the nanoparticle core and extending outward from the nanoparticle core. Oligonucleotide dendrons of the disclosure are nucleic acid structures comprising an oligonucleotide stem to which a plurality of oligonucleotide branches is linked via one or more doubler moieties, trebler moieties, or a combination thereof. See, e.g., FIG. 1A. Thus, an oligonucleotide dendron of the disclosure is a single oligonucleotide molecule having a dendritic architecture. In various embodiments, an oligonucleotide dendron and/or an oligonucleotide that is not a dendron further comprises an additional agent (e.g., a protein, a small molecule, a peptide, or a combination thereof). Thus, the disclosure also contemplates, in various aspects and embodiments, use of oligonucleotides that are not oligonucleotide dendrons (i.e., oligonucleotides that are not linked to other oligonucleotides via doubler moieties, trebler moieties, or a combination thereof). Accordingly, in some embodiments, a SNA comprising a nanoparticle core and an oligonucleotide dendron attached to the external surface of the nanoparticle core further comprises one or more oligonucleotides that are not oligonucleotide dendrons that are also attached to the nanoparticle core. It will be understood that all features of oligonucleotides described herein (e.g., type (DNA/RNA), single/double stranded, length, sequence, modified forms) apply to all oligonucleotides described herein, including oligonucleotide dendrons, oligonucleotide stems, oligonucleotide branches, and oligonucleotides that are not oligonucleotide dendrons.

In various embodiments, an oligonucleotide dendron comprises about 2 to about 27 branches. In further embodiments, an oligonucleotide dendron comprises about 2 to about 25, or about 2 to about 23, or about 2 to about 20, or about 2 to about 18, or about 2 to about 16, or about 2 to about 15, or about 2 to about 13, or about 2 to about 10, or about 2 to about 8, or about 2 to about 7, or about 2 to about 5, or about 2 to about 4, or about 2 to about 3 oligonucleotide branches. In further embodiments, an oligonucleotide dendron comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, or at least 27 oligonucleotide branches. In further embodiments, an oligonucleotide dendron comprises less than 27, less than 26, less than 25, less than 24, less than 23, less than 22, less than 21, less than 20, less than 19, less than 18, less than 17, less than 16, less than 15, less than 14, less than 13, less than 12, less than 11, less than 10, less than 9, less than 8, less than 7, less than 6, less than 5, less than 4, or less than 3 oligonucleotide branches. In some embodiments, an oligonucleotide dendron comprises or consists of 2, 3, 4, 5, 6, 7, 8, 9, 10, 11, 12, 13, 14, 15, 16, 17, 18, 19, 20, 21, 22, 23, 24, 25, 26, or 27 oligonucleotide branches. In further embodiments, an oligonucleotide dendron comprises 2, 3, 4, 6, 8, 9, 12, 18, or 27 oligonucleotide branches. In still further embodiments, an oligonucleotide dendron consists of 2, 3, 4, 6, 8, 9, 12, 18, or 27 oligonucleotide branches. In some embodiments, an oligonucleotide dendron consists of 6 branches. In some embodiments, an oligonucleotide dendron consists of 9 branches. In some embodiments, a SNA of the disclosure comprises two or more oligonucleotide dendrons. In some embodiments, each oligonucleotide dendron attached to the SNA comprises the same number of oligonucleotide branches. In some embodiments, at least two of the oligonucleotide dendrons attached to the SNA comprises a different number of oligonucleotide branches relative to each other.

Oligonucleotides (e.g., an oligonucleotide dendron, an oligonucleotide stem, an oligonucleotide branch, or an oligonucleotide that is not a dendron) contemplated for use according to the disclosure include, in various embodiments, DNA oligonucleotides, RNA oligonucleotides, modified forms thereof, or a combination thereof. Thus, in some embodiments, the oligonucleotide dendron is a DNA dendron, a RNA dendron, a modified oligonucleotide dendron, or a combination thereof. In some embodiments, the oligonucleotide stem is RNA and each oligonucleotide branch that is attached to the oligonucleotide stem through a doubler moiety, a trebler moiety, or a combination thereof is DNA. In some embodiments, the oligonucleotide stem is DNA and each oligonucleotide branch that is attached to the oligonucleotide stem through a doubler moiety, a trebler moiety, or a combination thereof is RNA. Thus, in any of the aspects or embodiments of the disclosure, the oligonucleotide stem portion of an oligonucleotide dendron may be a different nucleic acid class than the oligonucleotide branches that are attached to the oligonucleotide stem through a doubler moiety, a trebler moiety, or a combination thereof, but each oligonucleotide branch in the oligonucleotide dendron is the same nucleic acid class (e.g., the oligonucleotide stem can be DNA while each oligonucleotide branch is RNA).

In any aspects or embodiments described herein, an oligonucleotide is single-stranded, double-stranded, or partially double-stranded. Thus, in various embodiments, oligonucleotide stems and oligonucleotide branches can be single, double, or partially double stranded. In some embodiments, the oligonucleotide stem is used to hybridize the oligonucleotide dendron to a core structure to help form a DNA dendrimer, while the oligonucleotide branches remain unhybridized. Similarly, in some embodiments the oligonucleotide branches are used to hybridize to complementary structures. Modified forms of oligonucleotides are also contemplated which include those having at least one modified internucleotide linkage. In some embodiments, the oligonucleotide is all or in part a peptide nucleic acid. Other modified internucleoside linkages include at least one phosphorothioate linkage. Still other modified oligonucleotides include those comprising one or more universal bases. “Universal base” refers to molecules capable of substituting for binding to any one of A, C, G, T and U in nucleic acids by forming hydrogen bonds without significant structure destabilization. The oligonucleotide incorporated with the universal base analogues is able to function, e.g., as a probe in hybridization. Examples of universal bases include but are not limited to 5′-nitroindole-2′-deoxyriboside, 3-nitropyrrole, inosine and hypoxanthine.

The term “nucleotide” or its plural as used herein is interchangeable with modified forms as discussed herein and otherwise known in the art. The term “nucleobase” or its plural as used herein is interchangeable with modified forms as discussed herein and otherwise known in the art. Nucleotides or nucleobases comprise the naturally occurring nucleobases A, G, C, T, and U. Non-naturally occurring nucleobases include, for example and without limitations, xanthine, diaminopurine, 8-oxo-N6-methyladenine, 7-deazaxanthine, 7-deazaguanine, N4,N4-ethanocytosin, N′,N′-ethano-2,6-diaminopurine, 5-methylcytosine (mC), 5-(C3-C6)-alkynyl-cytosine, 5-fluorouracil, 5-bromouracil, pseudoisocytosine, 2-hydroxy-5-methyl-4-tr-iazolopyridin, isocytosine, isoguanine, inosine and the “non-naturally occurring” nucleobases described in Benner et al., U.S. Pat. No. 5,432,272 and Susan M. Freier and Karl-Heinz Altmann, 1997, Nucleic Acids Research, vol. 25: pp 4429-4443. The term “nucleobase” also includes not only the known purine and pyrimidine heterocycles, but also heterocyclic analogues and tautomers thereof. Further naturally and non-naturally occurring nucleobases include those disclosed in U.S. Pat. No. 3,687,808 (Merigan, et al.), in Chapter 15 by Sanghvi, in Antisense Research and Application, Ed. S. T. Crooke and B. Lebleu, CRC Press, 1993, in Englisch et al., 1991, Angewandte Chemie, International Edition, 30: 613-722 (see especially pages 622 and 623, and in the Concise Encyclopedia of Polymer Science and Engineering, J. I. Kroschwitz Ed., John Wiley & Sons, 1990, pages 858-859, Cook, Anti-Cancer Drug Design 1991, 6, 585-607, each of which are hereby incorporated by reference in their entirety). In various aspects, oligonucleotides also include one or more “nucleosidic bases” or “base units” which are a category of non-naturally-occurring nucleotides that include compounds such as heterocyclic compounds that can serve like nucleobases, including certain “universal bases” that are not nucleosidic bases in the most classical sense but serve as nucleosidic bases. Universal bases include 3-nitropyrrole, optionally substituted indoles (e.g., 5-nitroindole), and optionally substituted hypoxanthine. Other desirable universal bases include, pyrrole, diazole or triazole derivatives, including those universal bases known in the art.

Examples of oligonucleotides include those containing modified backbones or non-natural internucleoside linkages. Oligonucleotides having modified backbones include those that retain a phosphorus atom in the backbone and those that do not have a phosphorus atom in the backbone. Modified oligonucleotides that do not have a phosphorus atom in their internucleoside backbone are considered to be within the meaning of “oligonucleotide”.

Modified oligonucleotide backbones containing a phosphorus atom include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Also contemplated are oligonucleotides having inverted polarity comprising a single 3′ to 3′ linkage at the 3′-most internucleotide linkage, i.e. a single inverted nucleoside residue which may be abasic (the nucleotide is missing or has a hydroxyl group in place thereof). Salts, mixed salts and free acid forms are also contemplated. Representative United States patents that teach the preparation of the above phosphorus-containing linkages include, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, the disclosures of which are incorporated by reference herein.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages; siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts. See, for example, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, the disclosures of which are incorporated herein by reference in their entireties.

In still further embodiments, oligonucleotide mimetics wherein both one or more sugar and/or one or more internucleotide linkage of the nucleotide units are replaced with “non-naturally occurring” groups. The bases of the oligonucleotide are maintained for hybridization.

In some aspects, this embodiment contemplates a peptide nucleic acid (PNA). In PNA compounds, the sugar-backbone of an oligonucleotide is replaced with an amide containing backbone. See, for example U.S. Pat. Nos. 5,539,082; 5,714,331; and 5,719,262, and Nielsen et al., Science, 1991, 254, 1497-1500, the disclosures of which are herein incorporated by reference.

In still further embodiments, oligonucleotides are provided with phosphorothioate backbones and oligonucleosides with heteroatom backbones, and including —CH₂—NH—O—CH₂—, —CH₂—N(CH₃)—O—CH₂—, —CH₂—O—N(CH₃)—CH₂—, —CH₂—N(CH₃)—N(CH₃)—CH₂— and —O—N(CH₃)—CH₂—CH₂— described in U.S. Pat. Nos. 5,489,677, and 5,602,240. Also contemplated are oligonucleotides with morpholino backbone structures described in U.S. Pat. No. 5,034,506.

In various forms, the linkage between two successive monomers in the oligonucleotide consists of 2 to 4, desirably 3, groups/atoms selected from —CH₂—, —O—, —S—, —NR^{H}—, >C═O, >C═NR^{H}, >C═S, —Si(R″)₂—, —SO—, —S(O)₂—, —P(O)₂—, —PO(BH₃)—, —P(O,S)—, —P(S)₂—, —PO(R″)—, —PO(OCH₃)—, and —PO(NHR^{H})—, where RH is selected from hydrogen and C₁₋₄-alkyl, and R″ is selected from C₁₋₆alkyl and phenyl. Illustrative examples of such linkages are —CH₂—CH₂—CH₂—, —CH₂—CO—CH₂—, —CH₂—CHOH—CH₂—, —O—CH₂—O—, —O—CH₂—CH₂—, —O—CH₂—CH═(including R⁵ when used as a linkage to a succeeding monomer), —CH₂—CH₂—O—, —NR^{H}—CH₂—CH₂—, —CH₂—CH₂—NR^{H}—, —CH₂—NR^{H}—CH₂—, —O—CH₂—CH₂—NR^{H}—, —NR^{H}—CO—O—, —NR^{H}—CO—NR^{H}, —NR^{H}—CS—NR^{H}—, —NR^{H}—C(═NR^{H})—NR^{H}—, —NR^{H}—CO—CH₂—NR^{H}—O—CO—O—, —O—CO—CH₂—O—, —O—CH₂—CO—O—, —CH₂—CO—NR^{H}—, —O—CO—NR^{H}—, —NR^{H}—CO—CH₂—, —O—CH₂—CO—NR^{H}—, —O—CH₂—CH₂—NR^{H}—, —CH═N—O—, —CH₂—NR^{H}—O—, —CH₂—O—N=(including R⁵ when used as a linkage to a succeeding monomer), —CH₂—O—NR^{H}—, —CO—NR^{H}— CH₂—, —CH₂—NR^{H}—O—, —CH₂—NR^{H}—CO—, —O—NR^{H}— CH₂—, —O—NR^{H}, —O— CH₂—S—, —S— CH₂—O—, —CH₂— CH₂—S—, —O—CH₂— CH₂—S—, —S—CH₂—CH═(including R⁵ when used as a linkage to a succeeding monomer), —S—CH₂— CH₂—, —S—CH₂— CH₂—O—, —S—CH₂— CH₂—S—, —CH₂—S—CH₂—, —CH₂—SO— CH₂—, —CH₂—SO₂— CH₂—, —O—SO—O—, —O—S(O)₂—O—, —O—S(O)₂— CH₂—, —O—S(O)₂—NR^{H}—, —NR″—S(O)₂— CH₂—; —O—S(O)₂— CH₂—, —O—P(O)₂—O—, —O—P(O,S)—O—, —O—P(S)₂—O—, —S—P(O)₂—O—, —S—P(O,S)—O—, —S—P(S)₂—O—, —O—P(O)₂—S—, —O—P(O,S)—S—, —O—P(S)₂—S—, —S—P(O)₂—S—, —S—P(O,S)—S—, —S—P(S)₂—S—, —O—PO(R″)—O—, —O—PO(OCH₃)—O—, —O—PO(O CH₂CH₃)—O—, —O—PO(O CH₂CH₂S—R)—O—, —O—PO(BH₃)—O—, —O—PO(NHR^{N})—O—, —O—P(O)₂—NR^{H} H—, —NR^{H}—P(O)₂—O—, —O—P(O,NR^{H})—CH₂—P(O)₂—O—, —O—P(O)₂— CH₂—, and —O—Si(R″)₂—O—; among which —CH₂—CO—NR^{H}—, —CH₂—NR^{H}—O—, —S—CH₂—O—, —O—P(O)₂—O—O—P(— O,S)—O—, —O—P(S)₂—O—, —NR^{H} P(O)₂—O—, —O—P(O,NR^{H})—O—, —O—PO(R″)—O—, —O—PO(CH₃)—O—, and —O—PO(NHR^{N})—O—, where RH is selected form hydrogen and C₁₋₄-alkyl, and R″ is selected from C₁₋₆alkyl and phenyl, are contemplated. Further illustrative examples are given in Mesmaeker et. al., Current Opinion in Structural Biology 1995, 5, 343-355 and Susan M. Freier and Karl-Heinz Altmann, Nucleic Acids Research, 1997, vol 25, pp 4429-4443.

Still other modified forms of oligonucleotides are described in detail in U.S. patent application No. 20040219565, the disclosure of which is incorporated by reference herein in its entirety.

Modified oligonucleotides may also contain one or more substituted sugar moieties. In certain aspects, oligonucleotides comprise one of the following at the 2′ position: OH; F; O—, S—, or N-alkyl; O—, S—, or N-alkenyl; O—, S— or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Other embodiments include O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃]₂, where n and m are from 1 to about 10. Other oligonucleotides comprise one of the following at the 2′ position: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, or an RNA cleaving group. In one aspect, a modification includes 2′-methoxyethoxy (2′—O—CH₂CH₂OCH₃, also known as 2′—O-(2-methoxyethyl) or 2′-MOE) (Martin et al., Helv. Chim. Acta, 1995, 78, 486-504) i.e., an alkoxyalkoxy group. Other modifications include 2′-dimethylaminooxyethoxy, i.e., a O(CH₂)₂ON(CH₃)₂ group, also known as 2′-DMAOE, and 2′-dimethylaminoethoxyethoxy (also known in the art as 2′—O-dimethyl-amino-ethoxy-ethyl or 2′-DMAEOE), i.e., 2′—O—CH₂—O—CH₂—N(CH₃)₂.

Still other modifications include 2′-methoxy (2′—O—CH₃), 2′-aminopropoxy (2′-OCH₂CH₂CH₂NH₂), 2′-allyl (2′—CH₂—CH═CH₂), 2′—O-allyl (2′—O—CH₂—CH═CH₂) and 2′-fluoro (2′-F). The 2′-modification may be in the arabino (up) position or ribo (down) position. In one aspect, a 2′-arabino modification is 2′-F. Similar modifications may also be made at other positions on the oligonucleotide, for example, at the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. See, for example, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, the disclosures of which are incorporated by reference in their entireties herein.

In some aspects, a modification of the sugar includes Locked Nucleic Acids (LNAs) in which the 2′-hydroxyl group is linked to the 3′ or 4′ carbon atom of the sugar ring, thereby forming a bicyclic sugar moiety. The linkage is in certain aspects is a methylene (—CH₂—)ₙ group bridging the 2′ oxygen atom and the 4′ carbon atom wherein n is 1 or 2. LNAs and preparation thereof are described in WO 98/39352 and WO 99/14226.

Modified nucleotides are described in EP 1 072 679 and WO 97/12896, the disclosures of which are incorporated herein by reference. Modified nucleobases include without limitation, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified bases include tricyclic pyrimidines such as phenoxazine cytidine(1 H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzox-azin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified bases may also include those in which the purine or pyrimidine base is replaced with other heterocycles, for example 7-deaza-adenine, 7-deazaguanosine, 2-aminopyridine and 2-pyridone. Additional nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., 1991, Angewandte Chemie, International Edition, 30: 613, and those disclosed by Sanghvi, Y. S., Chapter 15, Antisense Research and Applications, pages 289-302, Crooke, S. T. and Lebleu, B., ed., CRC Press, 1993. Certain of these bases are useful for increasing binding affinity and include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. and are, in certain aspects combined with 2′—O-methoxyethyl sugar modifications. See, U.S. Pat. Nos. 3,687,808, 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; 5,750,692 and 5,681,941, the disclosures of which are incorporated herein by reference.

Methods of making polynucleotides of a predetermined sequence are well-known. See, e.g., Sambrook et al., Molecular Cloning: A Laboratory Manual (2nd ed. 1989) and F. Eckstein (ed.) Oligonucleotides and Analogues, 1st Ed. (Oxford University Press, New York, 1991). Solid-phase synthesis methods are preferred for both polyribonucleotides and polydeoxyribonucleotides (the well-known methods of synthesizing DNA are also useful for synthesizing RNA). Polyribonucleotides can also be prepared enzymatically. Non-naturally occurring nucleobases can be incorporated into the polynucleotide, as well. See, e.g., U.S. Pat. No. 7,223,833; Katz, J. Am. Chem. Soc., 74:2238 (1951); Yamane, et al., J. Am. Chem. Soc., 83:2599 (1961); Kosturko, et al., Biochemistry, 13:3949 (1974); Thomas, J. Am. Chem. Soc., 76:6032 (1954); Zhang, et al., J. Am. Chem. Soc., 127:74-75 (2005); and Zimmermann, et al., J. Am. Chem. Soc., 124:13684-13685 (2002).

In various aspects, an oligonucleotide of the disclosure, or a modified form thereof, is generally about 10 nucleotides to about 100 nucleotides in length. More specifically, an oligonucleotide of the disclosure is about 10 to about 90 nucleotides in length, about 10 to about 80 nucleotides in length, about 10 to about 70 nucleotides in length, about 10 to about 60 nucleotides in length, about 10 to about 50 nucleotides in length about 10 to about 45 nucleotides in length, about 10 to about 40 nucleotides in length, about 10 to about 35 nucleotides in length, about 10 to about 30 nucleotides in length, about 10 to about 25 nucleotides in length, about 10 to about 20 nucleotides in length, about 10 to about 15 nucleotides in length, and all oligonucleotides intermediate in length of the sizes specifically disclosed to the extent that the oligonucleotide is able to achieve the desired result. In further embodiments, an oligonucleotide of the disclosure is about 5 nucleotides to about 1000 nucleotides in length. In further embodiments, an oligonucleotide of the disclosure is about 5 to about 900 nucleotides in length, about 5 to about 800 nucleotides in length, about 5 to about 700 nucleotides in length, about 5 to about 600 nucleotides in length, about 5 to about 500 nucleotides in length about 5 to about 450 nucleotides in length, about 5 to about 400 nucleotides in length, about 5 to about 350 nucleotides in length, about 5 to about 300 nucleotides in length, about 5 to about 250 nucleotides in length, about 5 to about 200 nucleotides in length, about 5 to about 150 nucleotides in length, about 5 to about 100 nucleotides in length, about 5 to about 90 nucleotides in length, about 5 to about 80 nucleotides in length, about 5 to about 70 nucleotides in length, about 5 to about 60 nucleotides in length, about 5 to about 50 nucleotides in length, about 5 to about 40 nucleotides in length, about 5 to about 30 nucleotides in length, about 5 to about 20 nucleotides in length, about 5 to about 10 nucleotides in length, and all oligonucleotides intermediate in length of the sizes specifically disclosed to the extent that the oligonucleotide is able to achieve the desired result. Accordingly, in various embodiments, an oligonucleotide of the disclosure is or is at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900, 1000, or more nucleotides in length. In further embodiments, an oligonucleotide of the disclosure is less than 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900, 1000, or more nucleotides in length. In further embodiments, an oligonucleotide stem of the disclosure is about 1-50 nucleotides, about 1-40 nucleotides, about 1-30 nucleotides, about 1-20 nucleotides, about 1-10 nucleotides, about 5-50 nucleotides, about 5-40 nucleotides, about 5-35 nucleotides, about 5-30 nucleotides, about 5-25 nucleotides, about 5-20 nucleotides, about 5-10 nucleotides, about 10-15 nucleotides, about 10-20 nucleotides, about 10-25 nucleotides, or about 10-30 nucleotides in length. In some embodiments, an oligonucleotide stem of the disclosure is or is about 15 nucleotides in length. In further embodiments, an oligonucleotide branch of the disclosure is about 1-30 nucleotides, about 1-25, about 1-20 nucleotides, about 1-15 nucleotides, about 1-10 nucleotides, about 1-5 nucleotides, about 5-10 nucleotides, about 5-15 nucleotides, about 5-20 nucleotides, about 5-25 nucleotides, about 5-30 nucleotides, about 10-15 nucleotides, about 10-20 nucleotides, about 10-25 nucleotides, or about 10-30 nucleotides in length. In some embodiments, an oligonucleotide branch of the disclosure is or is about 10 nucleotides in length.

In some embodiments, the oligonucleotide is an aptamer. Accordingly, all features and aspects of oligonucleotides described herein (e.g., length, type (DNA, RNA, modified forms thereof), optional presence of spacer) also apply to aptamers. Aptamers are oligonucleotide sequences that can be evolved to bind to various target analytes of interest. Aptamers may be single stranded, double stranded, or partially double stranded.

Methods of attaching detectable markers (e.g., fluorophores, radiolabels) and additional moieties (e.g., an antibody) as described herein to an oligonucleotide are known in the art.

Nanoparticle surface density. A surface density adequate to make the nanoparticles stable and the conditions necessary to obtain it for a desired combination of nanoparticles and oligonucleotides can be determined empirically. In some embodiments, one oligonucleotide dendron is attached to the external surface of a nanoparticle core. In further embodiments, about 2 to about 100 oligonucleotide dendrons are attached to the external surface of a nanoparticle core. In further embodiments, about 2 to about 90, or about 2 to about 80, or about 2 to about 70, or about 2 to about 60, or about 2 to about 50, or about 2 to about 40, or about 2 to about 30, or about 2 to about 20, or about 2 to about 10, or about 10 to about 100, or about 10 to about 90, or about 10 to about 80, or about 10 to about 70, or about 10 to about 60, or about 10 to about 50, or about 10 to about 40, or about 10 to about 30, or about 10 to about 20, or about 20 to about 100, or about 20 to about 90, or about 20 to about 80, or about 20 to about 70, or about 20 to about 60, or about 20 to about 50, or about 20 to about 40, or about 20 to about 30 oligonucleotide dendrons are attached to the external surface of a nanoparticle core. When the nanoparticle core is a protein core, it is contemplated that in some embodiments about 1 to about 10 oligonucleotide dendrons are attached to the protein core. In further embodiments, 1, 2, 3, 4, 5, 6, 7, 8, 9, or 10 oligonucleotide dendrons are attached to the protein core. In still further embodiments, at least 1, at least 2, at least 3, at least 4, at least 5, at least 6, at least 7, at least 8, at least 9, or at least 10 oligonucleotide dendrons are attached to the protein core. In some embodiments, 1, 2, 3, 4, or 5 oligonucleotide dendrons are attached to a peptide core.

As described herein, in some embodiments a SNA comprises one or more oligonucleotides attached to the nanoparticle core that are not oligonucleotide dendrons. In some embodiments, oligonucleotides that are not oligonucleotide dendrons are attached to the nanoparticle core at a surface density of at least about 2 pmoles/cm². In some aspects, the surface density is at least 15 pmoles/cm². In some embodiments, oligonucleotide dendrons are attached to the nanoparticle core at a surface density of at least about 2 pmoles/cm². In some aspects, the surface density is at least 15 pmoles/cm². Methods are also provided wherein the oligonucleotide dendron and/or the oligonucleotide that is not an oligonucleotide dendron is bound to the nanoparticle at a surface density of at least 2 pmol/cm², at least 3 pmol/cm², at least 4 pmol/cm², at least 5 pmol/cm², at least 6 pmol/cm², at least 7 pmol/cm², at least 8 pmol/cm², at least 9 pmol/cm², at least 10 pmol/cm², at least about 15 pmol/cm², at least about 19 pmol/cm², at least about 20 pmol/cm², at least about 25 pmol/cm², at least about 30 pmol/cm², at least about 35 pmol/cm², at least about 40 pmol/cm², at least about 45 pmol/cm², at least about 50 pmol/cm², at least about 55 pmol/cm², at least about 60 pmol/cm², at least about 65 pmol/cm², at least about 70 pmol/cm², at least about 75 pmol/cm², at least about 80 pmol/cm², at least about 85 pmol/cm², at least about 90 pmol/cm², at least about 95 pmol/cm², at least about 100 pmol/cm², at least about 125 pmol/cm², at least about 150 pmol/cm², at least about 175 pmol/cm², at least about 200 pmol/cm², at least about 250 pmol/cm², at least about 300 pmol/cm², at least about 350 pmol/cm², at least about 400 pmol/cm², at least about 450 pmol/cm², at least about 500 pmol/cm², at least about 550 pmol/cm², at least about 600 pmol/cm², at least about 650 pmol/cm², at least about 700 pmol/cm², at least about 750 pmol/cm², at least about 800 pmol/cm², at least about 850 pmol/cm², at least about 900 pmol/cm², at least about 950 pmol/cm², at least about 1000 pmol/cm² or more. Alternatively, the density of oligonucleotides attached to the SNA that are not oligonucleotide dendrons is measured by the number of oligonucleotides attached to the SNA. With respect to the surface density of oligonucleotides attached to an SNA that are not oligonucleotide dendrons, it is contemplated that a SNA as described herein comprises about 1 to about 2,500, or about 1 to about 500 oligonucleotides on its surface. In various embodiments, a SNA comprises about 10 to about 500, or about 10 to about 300, or about 10 to about 200, or about 10 to about 190, or about 10 to about 180, or about 10 to about 170, or about 10 to about 160, or about 10 to about 150, or about 10 to about 140, or about 10 to about 130, or about 10 to about 120, or about 10 to about 110, or about 10 to about 100, or 10 to about 90, or about 10 to about 80, or about 10 to about 70, or about 10 to about 60, or about 10 to about 50, or about 10 to about 40, or about 10 to about 30, or about 10 to about 20 oligonucleotides in the shell of oligonucleotides attached to the nanoparticle core. In some embodiments, a SNA comprises about 80 to about 140 oligonucleotides in the shell of oligonucleotides attached to the nanoparticle core. In further embodiments, a SNA comprises at least about 5, 10, 20, 30, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200 oligonucleotides in the shell of oligonucleotides attached to the nanoparticle core. In further embodiments, a SNA consists of 1, 2, 3, 4, 5, 10, 20, 30, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200 oligonucleotides in the shell of oligonucleotides attached to the nanoparticle core. In still further embodiments, the shell of oligonucleotides attached to the nanoparticle core of the SNA comprises 2, 3, 4, 5, 6, 7, 8, 9, 10, 11, 12, 13, 14, 15, 16, 17, 18, 19, 20 or more oligonucleotides. In some embodiments, the shell of oligonucleotides attached to the nanoparticle core of the SNA comprises at least 20 oligonucleotides. In some embodiments, the shell of oligonucleotides attached to the nanoparticle core of the SNA consists of 1, 2, 3, 4, 5, 6, 7, 8, 9, 10, 11, 12, 13, 14, 15, 16, 17, 18, 19, or 20 oligonucleotides. In various embodiments of the disclosure, the number of dendron and non-dendron oligonucleotides attached to a nanoparticle core can be varied depending on the ratio of dendron to non-dendron oligonucleotides is added to the nanoparticle. If the maximum number of oligonucleotides to be attached to a nanoparticle core is 200, then in some embodiments 20 dendron oligonucleotides and 180 non-dendron oligonucleotides, or 40 dendron oligonucleotides and 160 non dendron oligonucleotides, or 60 dendron oligonucleotides and 140 non dendron oligonucleotides, or 80 dendron oligonucleotides and 120 non-dendron oligonucleotides, or 100 dendron oligonucleotides and 100 non-dendron oligonucleotides, or 120 dendron oligonucleotides and 80 non-dendron oligonucleotides, or 140 dendron oligonucleotides and 60 non dendron oligonucleotides, or 160 dendron oligonucleotides and 40 non dendron oligonucleotides, or 180 dendron oligonucleotides and 20 non dendron oligonucleotides.

Spacers. In some aspects, an oligonucleotide (e.g., an oligonucleotide stem) is attached to a nanoparticle through a spacer (and, in some embodiments, additionally through a linker). In some embodiments, an oligonucleotide branch is attached to a doubler and/or a trebler moiety through a spacer. “Spacer” as used herein means a moiety that serves to increase distance between the nanoparticle and the oligonucleotide, or to increase distance between individual oligonucleotides when attached to the nanoparticle in multiple copies. Thus, spacers are contemplated being located between individual oligonucleotides in tandem, whether the oligonucleotides have the same sequence or have different sequences.

In some aspects, the spacer when present is an organic moiety. In some aspects, the spacer is a polymer, including but not limited to a water-soluble polymer, a nucleic acid, a polypeptide, an oligosaccharide, a carbohydrate, a lipid, an ethylglycol, or a combination thereof. In any of the aspects or embodiments of the disclosure, the spacer is an oligo(ethylene glycol)-based spacer. In various embodiments, an oligonucleotide comprises 1, 2, 3, 4, 5, or more spacer (e.g., Spacer-18 (hexaethyleneglycol)) moieties. In further embodiments, the spacer is an alkane-based spacer (e.g., C₁₂). In some embodiments, the spacer is an oligonucleotide spacer (e.g., T5). An oligonucleotide spacer may have any sequence that does not interfere with the ability of the oligonucleotide to perform an intended function (e.g., inhibit gene expression). In certain aspects, the bases of the oligonucleotide spacer are all adenylic acids, all thymidylic acids, all cytidylic acids, all guanylic acids, all uridylic acids, or all some other modified base.

In various embodiments, the length of the spacer is or is equivalent to at least about 2 nucleotides, at least about 3 nucleotides, at least about 4 nucleotides, at least about 5 nucleotides, 5-10 nucleotides, 10 nucleotides, 20 nucleotides, 10-30 nucleotides, or even greater than 30 nucleotides.

Oliqonucleotide attachment to a nanoparticle core. Oligonucleotides contemplated for use according to the disclosure include those attached to a nanoparticle core through any means (e.g., covalent or non-covalent attachment). Regardless of the means by which the oligonucleotide is attached to the nanoparticle, attachment in various aspects is effected through a 5′ linkage, a 3′ linkage, some type of internal linkage, or any combination of these attachments. In some embodiments, the oligonucleotide is covalently attached to a nanoparticle. In further embodiments, the oligonucleotide is non-covalently attached to a nanoparticle.

Methods of attachment are known to those of ordinary skill in the art and are described in U.S. Publication No. 2009/0209629, which is incorporated by reference herein in its entirety. Methods of attaching RNA to a nanoparticle are generally described in PCT/US2009/65822, which is incorporated by reference herein in its entirety. Methods of associating oligonucleotides with a liposomal particle are described in PCT/US2014/068429, which is incorporated by reference herein in its entirety.

Methods of attaching oligonucleotides to a protein core are described, e.g., in U.S. Patent Application Publication No. 2017/0232109 and Brodin et al., J Am Chem Soc. 137(47): 14838-41 (2015), each of which is incorporated by reference herein in its entirety. In general, a polynucleotide can be modified at a terminus with an alkyne moiety, e.g., a DBCO-type moiety for reaction with the azide of the protein surface:

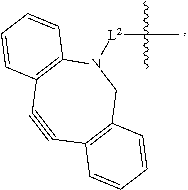

where L is a linker to a terminus of the polynucleotide. L² can be C₁₋₁₀ alkylene, —C(O)—C₁₋₁₀ alkylene-Y—, and —C(O)—C₁₋₁₀ alkylene-Y— C₁₋₁₀ alkylene-(OCH₂CH₂)ₘ—Y—; wherein each Y is independently selected from the group consisting of a bond, C(O), O, NH, C(O)NH, and NHC(O); and m is 0, 1, 2, 3, 4, or 5. For example, the DBCO functional group can be attached via a linker having a structure of

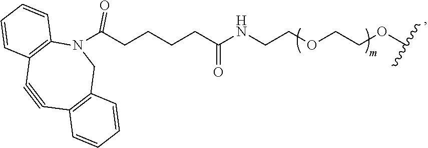

where the terminal “O” is from a terminal nucleotide on the polynucleotide. Use of this DBCO-type moiety results in a structure between the polynucleotide and the protein, in cases where a surface amine is modified, of:

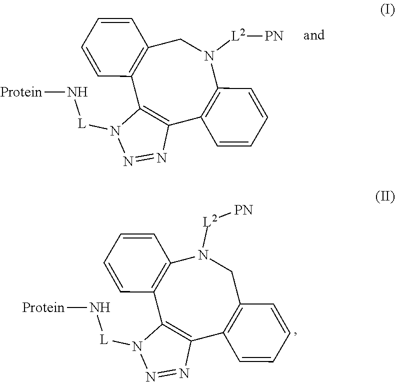

where L and L² are each independently selected from C₁₋₁₀ alkylene, —C(O)—C₁₋₁₀ alkylene-Y—, and —C(O)—C₁₋₁₀ alkylene-Y— C₁₋₁₀ alkylene-(OCH₂CH₂)ₘ—Y—; each Y is independently selected from the group consisting of a bond, C(O), O, NH, C(O)NH, and NHC(O); m is 0, 1, 2, 3, 4, or 5; and PN is the polynucleotide. Similar structures where a surface thiol or surface carboxylate of the protein are modified can be made in a similar fashion to result in comparable linkage structures.

The protein can be modified at a surface functional group (e.g., a surface amine, a surface carboxylate, a surface thiol) with a linker that terminates with an azide functional group: Protein-X-L-N₃, X is from a surface amino group (e.g., —NH—), carboxylic group (e.g., —C(O)— or —C(O)O—), or thiol group (e.g., —S—) on the protein; L is selected from C₁₋₁₀ alkylene, —Y—C(O)—C₁₋₁₀ alkylene-Y—, and —Y—C(O)—C₁₋₁₀ alkylene-Y— C₁₋₁₀ alkylene-(OCH₂CH₂)ₘ—Y—; each Y is independently selected from the group consisting of a bond, C(O), O, NH, C(O)NH, and NHC(O); and m is 0, 1, 2, 3, 4, or 5. Introduction of the “L-N₃” functional group to the surface moiety of the protein can be accomplished using well-known techniques. For example, a surface amine of the protein can be reacted with an activated ester of a linker having a terminal N₃ to form an amide bond between the amine of the protein and the carboxylate of the activated ester of the linker reagent.

The polynucleotide can be modified to include an alkyne functional group at a terminus of the polynucleotide: Polynucleotide-L₂-X—=—R; L² is selected from C₁₋₁₀ alkylene, —C(O)—C₁₋₁₀ alkylene-Y—, and —C(O)—C₁₋₁₀ alkylene-Y— C₁₋₁₀ alkylene-(OCH₂CH₂)ₘ—Y—; each Y is independently selected from the group consisting of a bond, C(O), O, NH, C(O)NH, and NHC(O); m is 0, 1, 2, 3, 4, or 5; and X is a bond and R is H or C₁₋₁₀alkyl; or X and R together with the carbons to which they are attached form a 8-10 membered carbocyclic or 8-10 membered heterocyclic group. In some cases, the polynucleotide has a structure

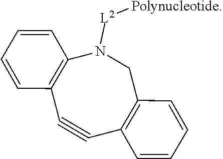

The protein, with the surface modified azide, and the polynucleotide, with a terminus modified to include an alkyne, can be reacted together to form a triazole ring in the presence of a copper (II) salt and a reducing agent to generate a copper (I) salt in situ. In some cases, a copper (I) salt is directly added. Contemplated reducing agents include ascorbic acid, an ascorbate salt, sodium borohydride, 2-mercaptoethanol, dithiothreitol (DTT), hydrazine, lithium aluminum hydride, diisobutylaluminum hydride, oxalic acid, Lindlar catalyst, a sulfite compound, a stannous compound, a ferrous compound, sodium amalgam, tris(2-carboxyethyl)phosphine, hydroquinone, and mixtures thereof.

The surface functional group of the protein can be attached to the polynucleotide using other attachment chemistries. For example, a surface amine can be directed conjugated to a carboxylate or activated ester at a terminus of the polynucleotide, to form an amide bond. A surface carboxylate can be conjugated to an amine on a terminus of the polynucleotide to form an amide bond. Alternatively, the surface carboxylate can be reacted with a diamine to form an amide bond at the surface carboxylate and an amine at the other terminus. This terminal amine can then be modified in a manner similar to that for a surface amine of the protein. A surface thiol can be conjugated with a thiol moiety on the polynucleotide to form a disulfide bond. Alternatively, the thiol can be conjugated with an activated ester on a terminus of a polynucleotide to form a thiocarboxylate.

Oligonucleotide features. SNAs of the disclosure comprise an oligonucleotide dendron that comprises an oligonucleotide stem linked to a plurality of oligonucleotide branches through a doubler moiety, a trebler moiety, or a combination thereof. As a result, in some embodiments, each SNA has the ability to bind to a plurality of target polynucleotides having a sequence sufficiently complementary to the target polynucleotide to hybridize under the conditions being used. For example, if a specific polynucleotide is targeted, a single SNA has the ability to bind to multiple copies of the same molecule. In some embodiments, methods are provided wherein the SNA comprises an oligonucleotide dendron having identical oligonucleotide branches, i.e., each oligonucleotide branch has the same length and the same sequence. In other aspects, the SNA comprises an oligonucleotide dendron having oligonucleotide branches that are not identical, i.e., at least one of the oligonucleotide branches of an oligonucleotide dendron differs from at least one other oligonucleotide branch of the oligonucleotide dendron in that it has a different length and/or a different sequence. In further embodiments, the SNA comprises two or more oligonucleotide dendrons, wherein each oligonucleotide dendron comprises oligonucleotide branches that are all the same, or one or more oligonucleotide branches differ from the other oligonucleotide branches in length and/or sequence. In aspects wherein a SNA comprises different oligonucleotide dendrons, the different oligonucleotide dendrons comprise, in various embodiments, oligonucleotide branches that bind to the same single target polynucleotide but at different locations, or bind to different target polynucleotides that encode different gene products. Accordingly, in various aspects, a single SNA may be used in a method to inhibit expression of more than one gene product. As described herein, in some embodiments a SNA further comprises one or more oligonucleotides that are not oligonucleotide dendrons. Such oligonucleotides that are not oligonucleotide dendrons may be inhibitory oligonucleotides as described herein. In some embodiments, the oligonucleotide stem of the oligonucleotide dendron is an inhibitory oligonucleotide as described herein. In some embodiments, one or more oligonucleotide branches of the oligonucleotide dendron is an inhibitory oligonucleotide as described herein. In some embodiments, the oligonucleotide stem and one or more oligonucleotide branches of the oligonucleotide dendron are immunostimulatory oligonucleotides as described herein. Thus, in various aspects, oligonucleotides (e.g., an oligonucleotide branch, an oligonucleotide stem, or an oligonucleotide that is not a dendron) are used to target specific polynucleotides, whether at one or more specific regions in the target polynucleotide, or over the entire length of the target polynucleotide as the need may be to effect a desired level of inhibition of gene expression.

In some embodiments, one or more oligonucleotide branches of the oligonucleotide dendron is an immunostimulatory oligonucleotide as described herein. In some embodiments, the oligonucleotide stem of the oligonucleotide dendron is an immunostimulatory oligonucleotide as described herein. In some embodiments, the oligonucleotide stem and one or more oligonucleotide branches of the oligonucleotide dendron is an immunostimulatory oligonucleotide as described herein. As described herein, in some embodiments a SNA further comprises one or more oligonucleotides that are not oligonucleotide dendrons. In some embodiments, such oligonucleotides that are not oligonucleotide dendrons are immunostimulatory oligonucleotides as described herein.

### Nanoparticle Core

One advantage of the present disclosure is that a single oligonucleotide dendron attached to a nanoparticle core can provide the nanoparticle core with SNA properties (e.g., high cellular uptake, resistance to degradation, ability to modulate an immune response). In general, nanoparticles contemplated by the disclosure include any compound or substance with a loading capacity for an oligonucleotide as described herein, including for example and without limitation, a protein, a metal, a semiconductor, a liposomal particle, a polymer-based particle (e.g., a poly (lactic-co-glycolic acid) (PLGA) particle), insulator particle compositions, and a dendrimer (organic versus inorganic). Thus, in various embodiments, the nanoparticle core is organic (e.g., a liposome), inorganic (e.g., gold, silver, or platinum), porous (e.g., silica-based or metal organic-framework-based), or hollow. In any of the aspects or embodiments of the disclosure, the nanoparticle core is a protein core. In some embodiments, a nanoparticle core is a peptide core or a small molecule core (including a drug core).

Thus, the disclosure contemplates nanoparticle cores that comprise a variety of inorganic materials including, but not limited to, metals, semi-conductor materials or ceramics as described in U.S. Patent Publication No 20030147966. For example, metal-based nanoparticles include those described herein. In various embodiments, the nanoparticle core is a metallic core, a semiconductor core, an insulator core, an upconverting core, a micellar core, a dendrimer core, a liposomal core, a polymer core, a metal-organic framework core, a protein core, or a combination thereof. Ceramic nanoparticle materials include, but are not limited to, brushite, tricalcium phosphate, alumina, silica, and zirconia. Organic materials from which nanoparticles are produced include carbon. Nanoparticle polymers include polystyrene, silicone rubber, polycarbonate, polyurethanes, polypropylenes, polymethylmethacrylate, polyvinyl chloride, polyesters, polyethers, and polyethylene. Biodegradable, biopolymer (e.g., polypeptides such as BSA, polysaccharides, etc.), other biological materials (e.g., carbohydrates), and/or polymeric compounds are also contemplated for use in producing nanoparticles. In some embodiments, the polymer is polylactide, a polylactide-polyglycolide copolymer, a polycaprolactone, a polyacrylate, alginate, albumin, silica, polypyrrole, polythiophene, polyaniline, polyethylenimine, poly(methyl methacrylate), chitosan, or a related structure. In some embodiments, the polymer is poly(lactic-co-glycolic acid) (PLGA).

Liposomal particles, for example as disclosed in International Patent Application No. PCT/US2014/068429 and U.S. Pat. No. 10,792,251 (each of which is incorporated by reference herein in its entirety) are also contemplated by the disclosure. Hollow particles, for example as described in U.S. Patent Publication Number 2012/0282186 (incorporated by reference herein in its entirety) are also contemplated herein. Liposomes of the disclosure have at least a substantially spherical geometry, an internal side and an external side, and comprise a lipid bilayer. The lipid bilayer comprises, in various embodiments, a lipid from the phosphocholine family of lipids or the phosphoethanolamine family of lipids. In various embodiments, the lipid is 1,2-dioleoyl-sn-glycero-3-phosphocholine (DOPC), 1,2-dimyristoyl-sn-phosphatidylcholine (DMPC), 1-palmitoyl-2-oleoyl-sn-phosphatidylcholine (POPC), 1,2-distearoyl-sn-glycero-3-phospho-(1′-rac-glycerol) (DSPG), 1,2-dioleoyl-sn-glycero-3-phospho-(1′-rac-glycerol) (DOPG), 1,2-distearoyl-sn-glycero-3-phosphocholine (DSPC), 1,2-dipalmitoyl-sn-glycero-3-phosphocholine (DPPC), 1,2-di-(9Z-octadecenoyl)-sn-glycero-3-phosphoethanolamine (DOPE), 1,2-dihexadecanoyl-sn-glycero-3-phosphoethanolamine (DPPE), cardiolipin, lipid A, or a combination thereof.

In some embodiments, the nanoparticle is metallic, and in various aspects, the nanoparticle is a colloidal metal. Thus, in various embodiments, nanoparticles useful in the practice of the methods include metal (including for example and without limitation, gold, silver, platinum, aluminum, palladium, copper, cobalt, indium, nickel, or any other metal amenable to nanoparticle formation), semiconductor (including for example and without limitation, CdSe, CdS, and CdS or CdSe coated with ZnS) and magnetic (for example, ferromagnetite) colloidal materials. Other nanoparticles useful in the practice of the invention include, also without limitation, ZnS, ZnO, Ti, TiO₂, Sn, SnO₂, Si, SiO₂, Fe, Fe⁺⁴, Ag, Cu, Ni, Al, steel, cobalt-chrome alloys, Cd, titanium alloys, Agl, AgBr, Hgl₂, PbS, PbSe, ZnTe, CdTe, In₂S₃, In₂Se₃, Cd₃P₂, Cd₃As₂, InAs, and GaAs. Methods of making ZnS, ZnO, TiO₂, Agl, AgBr, Hgl₂, PbS, PbSe, ZnTe, CdTe, In₂S₃, In₂Se₃, Cd₃P₂, Cd₃As₂, InAs, and GaAs nanoparticles are also known in the art. See, e.g., Weller, Angew. Chem. Int. Ed. Engl., 32, 41 (1993); Henglein, Top. Curr. Chem., 143, 113 (1988); Henglein, Chem. Rev., 89, 1861 (1989); Brus, Appl. Phys. A., 53, 465 (1991); Bahncmann, in Photochemical Conversion and Storage of Solar Energy (eds. Pelizetti and Schiavello 1991), page 251; Wang and Herron, J. Phys. Chem., 95, 525 (1991); Olshavsky, et al., J. Am. Chem. Soc., 112, 9438 (1990); Ushida et al., J. Phys. Chem., 95, 5382 (1992). In some embodiments, the nanoparticle is an iron oxide nanoparticle. In further embodiments, the nanoparticle core is gold, silver, platinum, aluminum, palladium, copper, cobalt, indium, cadmium selenide, iron oxide, fullerene, metal-organic framework, zinc sulfide, or nickel.

Methods of making metal, semiconductor and magnetic nanoparticles are well-known in the art. See, for example, Schmid, G. (ed.) Clusters and Colloids (VCH, Weinheim, 1994); Hayat, M. A. (ed.) Colloidal Gold: Principles, Methods, and Applications (Academic Press, San Diego, 1991); Massart, R., IEEE Transactions On Magnetics, 17, 1247 (1981); Ahmadi, T. S. et al., Science, 272, 1924 (1996); Henglein, A. et al., J. Phys. Chem., 99, 14129 (1995); Curtis, A. C., et al., Angew. Chem. Int. Ed. Engl., 27, 1530 (1988). Preparation of polyalkylcyanoacrylate nanoparticles prepared is described in Fattal, et al., J. Controlled Release (1998) 53: 137-143 and U.S. Pat. No. 4,489,055. Methods for making nanoparticles comprising poly(D-glucaramidoamine)s are described in Liu, et al., J. Am. Chem. Soc. (2004) 126:7422-7423. Preparation of nanoparticles comprising polymerized methylmethacrylate (MMA) is described in Tondelli, et al., Nucl. Acids Res. (1998) 26:5425-5431, and preparation of dendrimer nanoparticles is described in, for example Kukowska-Latallo, et al., Proc. Natl. Acad. Sci. USA (1996) 93:4897-4902 (Starburst polyamidoamine dendrimers).

Suitable nanoparticles are also commercially available from, for example, Ted Pella, Inc. (gold), Amersham Corporation (gold) and Nanoprobes, Inc. (gold).

In any of the aspects or embodiments of the disclosure, the nanoparticle core is a protein. In further embodiments, the nanoparticle core is a peptide core. As used herein, protein is used interchangeably with “polypeptide” and refers to one or more polymers of amino acid residues. In various embodiments of the disclosure, a protein core comprises or consists of a single protein (i.e., a single polymer of amino acids), a multimeric protein, a peptide (e.g., a polymer of amino acids that between about 2 and 50 amino acids in length), or a synthetic fusion protein of two or more proteins. Synthetic fusion proteins include, without limitation, an expressed fusion protein (expressed from a single gene) and post-expression fusions where proteins are conjugated together chemically. Protein/oligonucleotide core-shell nanoparticles are also generally described in U.S. Patent Application Publication No. 2017/0232109, which is incorporated by reference herein in its entirety.

Proteins are understood in the art and include without limitation an enzyme, a therapeutic protein (e.g., adenosine deaminase, phosphatase and tensin homolog (PTEN), or interleukin-2 (IL-2)), a structural protein (e.g., actin), an antibody, a storage protein (e.g., ovalbumin), a transport protein (e.g., hemoglobin), a hormone (e.g., insulin), a receptor protein (e.g., G-Protein Coupled Receptors), a motor protein (e.g., kinesin, dynein, or myosin), an immunogenic protein (e.g., ovalbumin or a stimulator of interferon genes (STING) protein) or a fluorescent protein (e.g., green fluorescent protein (GFP), mutant neon green (mNeonGreen), or ruby red protein). In various embodiments, proteins contemplated by the disclosure include without limitation those having catalytic, signaling, therapeutic, or transport activity.

Proteins of the present disclosure may be either naturally occurring or non-naturally occurring. Proteins optionally include a spacer as described herein.

Naturally occurring proteins include without limitation biologically active proteins (including antibodies) that exist in nature or can be produced in a form that is found in nature by, for example, chemical synthesis or recombinant expression techniques. Thus, a protein core of the disclosure is or comprises, in some embodiments, an antibody. Naturally occurring proteins also include lipoproteins and post-translationally modified proteins, such as, for example and without limitation, glycosylated proteins. Antibodies contemplated for use in the methods and compositions of the present disclosure include without limitation antibodies that recognize and associate with a target molecule either in vivo or in vitro.

Structural proteins contemplated by the disclosure include without limitation actin, tubulin, collagen, and elastin.

Non-naturally occurring proteins contemplated by the present disclosure include but are not limited to synthetic proteins, as well as fragments, analogs and variants of naturally occurring or non-naturally occurring proteins as defined herein. Non-naturally occurring proteins also include proteins or protein substances that have D-amino acids, modified, derivatized, or non-naturally occurring amino acids in the D- or L-configuration and/or peptidomimetic units as part of their structure. The term “peptide” typically refers to short (e.g., about 2-50 amino acids in length) polypeptides/proteins. Non-naturally occurring proteins are prepared, for example, using an automated protein synthesizer or, alternatively, using recombinant expression techniques using a modified polynucleotide which encodes the desired protein.

As used herein a “fragment” of a protein is meant to refer to any portion of a protein smaller than the full-length protein or protein expression product. As used herein an “analog” refers to any of two or more proteins substantially similar in structure and having the same biological activity, but can have varying degrees of activity, to either the entire molecule, or to a fragment thereof. Analogs differ in the composition of their amino acid sequences based on one or more mutations involving substitution, deletion, insertion and/or addition of one or more amino acids for other amino acids. Substitutions can be conservative or non-conservative based on the physico-chemical or functional relatedness of the amino acid that is being replaced and the amino acid replacing it. As used herein a “variant” refers to a protein or analog thereof that is modified to comprise additional chemical moieties not normally a part of the molecule. Such moieties may modulate, for example and without limitation, the molecules solubility, absorption, and/or biological half-life. Moieties capable of mediating such effects are disclosed in Remington's Pharmaceutical Sciences (1980). Procedures for coupling such moieties to a molecule are well known in the art. In various aspects, proteins are modified by glycosylation, pegylation, and/or polysialylation.

Fusion proteins, including fusion proteins wherein one fusion component is a fragment or a mimetic, are also contemplated. A “mimetic” as used herein means a peptide or protein having a biological activity that is comparable to the protein of which it is a mimetic. By way of example, an endothelial growth factor mimetic is a peptide or protein that has a biological activity comparable to the native endothelial growth factor. The term further includes peptides or proteins that indirectly mimic the activity of a protein of interest, such as by potentiating the effects of the natural ligand of the protein of interest.

Proteins include antibodies along with fragments and derivatives thereof, including but not limited to Fab′ fragments, F(ab)₂ fragments, Fv fragments, Fc fragments, one or more complementarity determining regions (CDR) fragments, individual heavy chains, individual light chain, dimeric heavy and light chains (as opposed to heterotetrameric heavy and light chains found in an intact antibody, single chain antibodies (scAb), humanized antibodies (as well as antibodies modified in the manner of humanized antibodies but with the resulting antibody more closely resembling an antibody in a non-human species), chelating recombinant antibodies (CRABs), bispecific antibodies and multispecific antibodies, and other antibody derivative or fragments known in the art.

### SNA Synthesis

As described herein, one advantage of the oligonucleotide dendrimer SNAs of the present disclosure is that a single oligonucleotide dendron attached to the nanoparticle core of the SNA can effect delivery of, e.g., oligonucleotides, proteins, and small molecules. Thus, in some aspects, an oligonucleotide dendrimer SNA of the disclosure consists of one oligonucleotide dendron. SNAs of the disclosure are synthesized such that an oligonucleotide dendron is attached to the external surface of a nanoparticle core. The oligonucleotide dendron comprises an oligonucleotide stem to which a plurality of oligonucleotide branches is linked through a doubler moiety, a trebler moiety, or a combination thereof.

In general, and by way of example, oligonucleotide dendrons of the disclosure may be synthesized using an automated oligonucleotide synthesizer on 2000 angstrom controlled pore glass (CPG) beads, commonly used in solid-phase oligonucleotide synthesis. Oligonucleotide (e.g., DNA) synthesis involves a series of coupling steps performed on each nucleotide base added to the structure. This comprises (1) a coupling step which attaches a new base to the previous one, (2) a capping step which deactivates any unreacted material, (3) an oxidation step which forms the characteristic phosphate backbone of DNA, and (4) a detritylation step which prepares the newly added base for the next addition. To produce oligonucleotide dendrons, branching units (e.g., doubler moieties, trebler moieties, or a combination thereof), were added into this sequence of nucleotide bases at the desired location. As a result, a stem region is initially synthesized as desired, then the branching units are used to create a plurality of oligonucleotides, and finally the branches are synthesized following the same oligonucleotide (e.g., DNA) synthesis cycle. By changing the type and number of branching units used, an oligonucleotide dendron, with any number of branches, can be synthesized. In various embodiments, during synthesis, functional groups can be added to the 3′ end (stem) or the 5′ end (branches) such that the dendrons may be conjugated to a wide variety of nanoparticle types using either direct attachment or linkers, as described herein.

Additional description of SNA synthesis is provided in Example 1, below. Examples of doubler moieties that may be used in the synthesis of an oligonucleotide dendron are shown below and are available from Glen Research, Sterling, VA. Note that the structures below represent the doubler moiety prior to incorporation into the oligonucleotide dendron.

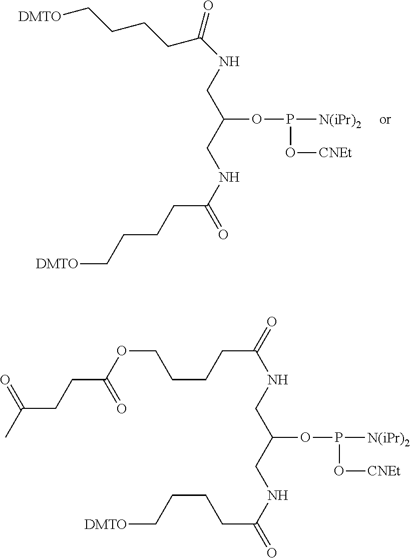

DMT=4,4′-dimethoxytriryl; O=oxygen; C═carbon; N=nitrogen; Et=ethyl; iPr=isopropyl

More generally, it is contemplated that in any of the aspects or embodiments of the disclosure a doubler moiety comprises the following structure:

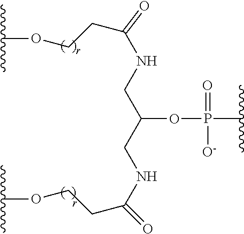 [SMILES: *OCCC(=O)NCC(CNC(=O)CCO*)OP(*)(=O)[O-]]

where each r can be 0, 1, 2, 3, 4, 5 or 6. P can be conjugated to an oligonucleotide portion of a dendron of the disclosure (e.g., oligonucleotide stem and/or oligonucleotide branch) or to an oxygen end group of another doubler moiety or trebler moiety and each oxygen end group of the oligonucleotide branches can be connected to a dendron or P of a further doubler or trebler. In some embodiments, each r=3.

Examples of trebler moieties that may be used in the synthesis of an oligonucleotide dendron are shown below and are available from Glen Research, Sterling, VA. Note that the structures below represent the trebler moiety prior to incorporation into the oligonucleotide dendron.

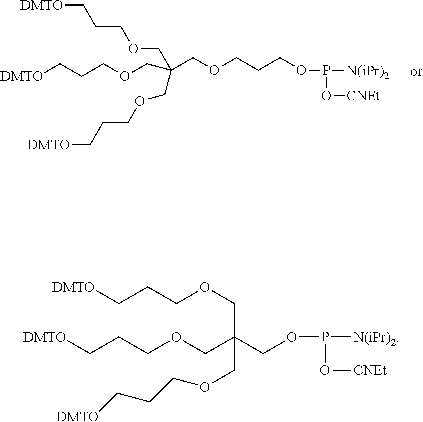

DMT=4,4′-dimethoxytriryl; O=oxygen; C═carbon; N=nitrogen; Et=ethyl; iPr=isopropyl

More generally, it is contemplated that in any of the aspects or embodiments of the disclosure a trebler moiety comprises the following structure:

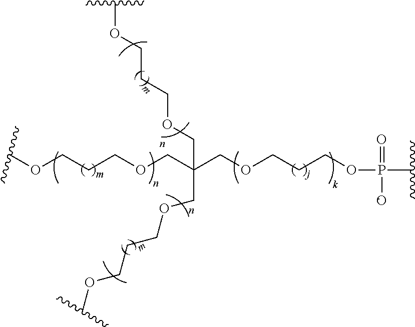

where each m can be 0, 1, 2, or 3; each n can be 1, 2, 3, or 4; j can be 0, 1, 2, or 3; and k can be 1, 2, 3, or 4. In some embodiments, j=1, k=1, each m=1 and each n=1.

### Use of SNAs in Gene Regulation/Therapy

It is contemplated that in any of the aspects or embodiments of the disclosure, a SNA as disclosed herein possesses the ability to regulate gene expression. Thus, in some embodiments, a SNA of the disclosure comprises an oligonucleotide dendron and/or an oligonucleotide that is not an oligonucleotide dendron having gene regulatory activity (e.g., inhibition of target gene expression or target cell recognition). In some embodiments, a SNA of the disclosure comprises an oligonucleotide dendron comprising an oligonucleotide stem that is an inhibitory oligonucleotide as described herein. In some embodiments, a SNA of the disclosure comprises an oligonucleotide dendron comprising one or more oligonucleotide branches that is an inhibitory oligonucleotide as described herein. In some embodiments, a SNA of the disclosure comprises an oligonucleotide dendron comprising an oligonucleotide stem and one or more oligonucleotide branches that are inhibitory oligonucleotides.

Accordingly, in some embodiments the disclosure provides methods for inhibiting gene product expression, and such methods include those wherein expression of a target gene product is inhibited by about or at least about 5%, about or at least about 10%, about or at least about 15%, about or at least about 20%, about or at least about 25%, about or at least about 30%, about or at least about 35%, about or at least about 40%, about or at least about 45%, about or at least about 50%, about or at least about 55%, about or at least about 60%, about or at least about 65%, about or at least about 70%, about or at least about 75%, about or at least about 80%, about or at least about 85%, about or at least about 90%, about or at least about 95%, about or at least about 96%, about or at least about 97%, about or at least about 98%, about or at least about 99%, or 100% compared to gene product expression in the absence of a SNA. In other words, methods provided embrace those which results in essentially any degree of inhibition of expression of a target gene product.

The degree of inhibition is determined in vivo from a body fluid sample or from a biopsy sample or by imaging techniques well known in the art. Alternatively, the degree of inhibition is determined in a cell culture assay, generally as a predictable measure of a degree of inhibition that can be expected in vivo resulting from use of a specific type of SNA and a specific oligonucleotide. In various aspects, the methods include use of an oligonucleotide branch sufficiently complementary to a target polynucleotide as described herein.

Accordingly, methods of utilizing a SNA of the disclosure in gene regulation therapy are provided. This method comprises the step of hybridizing a polynucleotide encoding the gene with one or more oligonucleotides (e.g., an oligonucleotide stem, an oligonucleotide branch and/or an oligonucleotide that is not an oligonucleotide dendron) complementary to all or a portion of the polynucleotide, wherein hybridizing between the polynucleotide and the oligonucleotide occurs over a length of the polynucleotide with a degree of complementarity sufficient to inhibit expression of the gene product. The inhibition of gene expression may occur in vivo or in vitro.

The inhibitory oligonucleotide utilized in the methods of the disclosure is either RNA, DNA, or a modified form thereof. In various embodiments, the inhibitory oligonucleotide is antisense DNA, small interfering RNA (siRNA), an aptamer, a short hairpin RNA (shRNA), a DNAzyme, or an aptazyme.

### Uses of SNAs in Immune Regulation

Toll-like receptors (TLRs) are a class of proteins, expressed in sentinel cells, that play a key role in regulation of innate immune system. The mammalian immune system uses two general strategies to combat infectious diseases. Pathogen exposure rapidly triggers an innate immune response that is characterized by the production of immunostimulatory cytokines, chemokines and polyreactive IgM antibodies. The innate immune system is activated by exposure to Pathogen Associated Molecular Patterns (PAMPs) that are expressed by a diverse group of infectious microorganisms. The recognition of PAMPs is mediated by members of the Toll-like family of receptors. TLR receptors, such as TLR 4, TLR 8 and TLR 9 that respond to specific oligonucleotide are located inside special intracellular compartments, called endosomes. The mechanism of modulation of, for example and without limitation, TLR 4, TLR 8 and TLR 9 receptors, is based on DNA-protein interactions.

Synthetic immunostimulatory oligonucleotides that contain CpG motifs that are similar to those found in bacterial DNA stimulate a similar response of the TLR receptors. Therefore, immunomodulatory oligonucleotides have various potential therapeutic uses, including treatment of immune deficiency and cancer. Thus, in some embodiments, a SNA of the disclosure comprises an oligonucleotide dendron comprising one or more oligonucleotide branches that is a TLR agonist. In some embodiments, a SNA of the disclosure comprises an oligonucleotide dendron comprising an oligonucleotide stem that is a TLR agonist. In some embodiments, a SNA of the disclosure comprises an oligonucleotide dendron comprising an oligonucleotide stem and one or more oligonucleotide branches that are TLR agonists. In some embodiments, a SNA of the disclosure comprises an oligonucleotide dendron comprising one or more oligonucleotide branches that is a TLR antagonist. In some embodiments, a SNA of the disclosure comprises an oligonucleotide dendron comprising an oligonucleotide stem that is a TLR antagonist. In some embodiments, a SNA of the disclosure comprises an oligonucleotide dendron comprising an oligonucleotide stem and one or more oligonucleotide branches that are TLR antagonists. In further embodiments, a SNA of the disclosure further comprises an oligonucleotide that is a TLR agonist, wherein the oligonucleotide is not an oligonucleotide dendron. In further embodiments, a SNA of the disclosure further comprises an oligonucleotide that is a TLR antagonist, wherein the oligonucleotide is not an oligonucleotide dendron. In some embodiments, the immunostimulatory oligonucleotide is a double-stranded DNA (dsDNA).

In further embodiments, down regulation of the immune system would involve knocking down the gene responsible for the expression of the Toll-like receptor. This antisense approach involves use of a SNA of the disclosure to knock down the expression of any toll-like protein.

Accordingly, in some embodiments, methods of utilizing SNAs as described herein for modulating toll-like receptors are disclosed. The method either up-regulates or down-regulates the Toll-like-receptor activity through the use of a TLR agonist or a TLR antagonist, respectively. The method comprises contacting a cell having a toll-like receptor with a SNA of the disclosure, thereby modulating the activity and/or the expression of the toll-like receptor. The toll-like receptors modulated include one or more of toll-like receptor 1, toll-like receptor 2, toll-like receptor 3, toll-like receptor 4, toll-like receptor 5, toll-like receptor 6, toll-like receptor 7, toll-like receptor 8, toll-like receptor 9, toll-like receptor 10, toll-like receptor 11, toll-like receptor 12, and/or toll-like receptor 13.

### Compositions

The disclosure also provides compositions that comprise a SNA of the disclosure, or a plurality thereof. In some embodiments, the composition further comprises a pharmaceutically acceptable carrier. The term “carrier” refers to a vehicle within which the SNA as described herein is administered to a subject. Any conventional media or agent that is compatible with the SNAs according to the disclosure can be used. The term carrier encompasses diluents, excipients, adjuvants and a combination thereof.

### Additional Agents

The SNAs provided herein optionally include an additional agent. The additional agent is, in various embodiments, simply associated with the oligonucleotide stem of an oligonucleotide dendron, one or more of the oligonucleotide branches of an oligonucleotide dendron, an oligonucleotide that is not an oligonucleotide dendron, and/or the additional agent is associated with the nanoparticle core of the SNA. In some embodiments, the additional agent is associated with the end of an oligonucleotide branch that is not connected to an oligonucleotide stem. In some embodiments, the additional agent is covalently associated with the oligonucleotide stem. In some embodiments, the additional agent is non-covalently associated with the oligonucleotide stem. It is contemplated that this additional agent is in one aspect covalently associated with the one or more of the plurality of oligonucleotide branches, or in the alternative, non-covalently associated with the one or more of the plurality of oligonucleotide branches. However, it is understood that the disclosure provides SNAs wherein one or more additional agents are both covalently and non-covalently associated with the oligonucleotide stem and/or the one or more of the plurality of oligonucleotide branches and/or an oligonucleotide that is not an oligonucleotide dendron. It will also be understood that non-covalent associations include hybridization, protein binding, and/or hydrophobic interactions.

Additional agents contemplated by the disclosure include without limitation a protein (e.g., a therapeutic protein), a small molecule, a peptide, or a combination thereof. These additional agents are described herein. Proteins and peptides are described herein and may be used as a nanoparticle core, an additional agent, or both.

The term “small molecule,” as used herein, refers to a chemical compound or a drug, or any other low molecular weight organic compound, either natural or synthetic. By “low molecular weight” is meant compounds having a molecular weight of less than 1500 Daltons, typically between 100 and 700 Daltons.

The following examples are given merely to illustrate the present disclosure and not in any way to limit its scope.

### EXAMPLES

The disclosure provides a robust, solid-phase synthesis for oligonucleotide dendrons that enables fine-tuned valency and density control by changing branching unit structure and dendron generation. As shown in the following examples, these dendrons were covalently conjugated onto single surface residues of proteins and peptides. Cellular uptake studies showed that uptake was achieved with just a single DNA dendron tag on the protein's surface. These findings confirmed that local DNA density rather than high surface density coverage dictated cellular uptake and as a result, DNA dendrons expand the scope of deliverable biomolecules to cells, while creating a biocompatible tag that can be used for the cellular delivery of many nanoscale materials.

### Example 1

### Synthesis of Oligonucleotide Dendron SNAs

Solid-phase phosphoramidite chemistry enables the synthesis of covalently linked structures that can be tailored, using branching units, to systematically increase ligand valency by altering the branching unit type and dendron generation [Shchepinov et al., Nucleic Acids Res. 1997, 25 (22), 4447-54]. However, covalent DNA dendrons typically suffer from low yields and low throughput of purified products.

Herein, a robust dendron synthesis and conjugation strategy is described, which harnesses automated DNA synthesis protocols and provides monodisperse branched DNA structures and protein conjugates. To access large monodisperse DNA dendrons with controlled numbers of branches, it was hypothesized that yields could be improved by increasing the bead pore size on controlled pore glass (CPG) solid supports and by decreasing steric hindrance through the introduction of a longer stem and flexible linkers. First, the limited volume for dendron growth was addressed by increasing the pore size of the CPG beads from 1000 A to 2000 A. This effectively decreased the number of DNA strands synthesized per bead because there was less surface area and enabled milder reaction conditions by allowing reagents to flow through the larger pores at lower pressures. Then, DNA steric hindrance and electrostatic repulsion were decreased through careful placement of hexaethylene glycol phosphoramidites near the branching units, which resulted in increased dendron molecular flexibility and spacing between DNA branches. Finally, CPG bead steric hindrance was addressed by increasing the dendron's stem from 5 bases to 15 bases, thus pushing it farther from the CPG surface and giving it more space to grow. This enabled the synthesis of DNA dendrons with size, length, and generation control, surpassing the size of those reported previously [Shchepinov et al., Nucleic Acids Res. 1997, 25 (22), 4447-54].

With these modifications, monodisperse DNA dendrons were synthesized with greater than 10% yield, an over 20-fold improvement compared to prior reports. Dendrons with various valences were accessed reliably and purified by denaturing polyacrylamide gel electrophoresis (PAGE). All dendrons were characterized by MALDI-TOF and denaturing PAGE to confirm structure and purity. Furthermore, functional groups, such as primary amines, could be introduced on the stem with no loss in yield, enabling dendron functionalization onto proteins.

CPG beads are measured and an amount that corresponds to a 10 umol DNA synthesis, as described on the CPG packaging, was added to a DNA synthesis column. 10 umol syntheses were conducted at a time. All reagents were added at a 10:1 molar ratio or higher. DNA dendrons are synthesized at room temperature under inert atmospheric conditions (argon). Nucleotides, including doubler and trebler moieties, are sequentially added, allowing for each addition to react for 5-12 minutes. Once the ABI synthesizer is complete, DNA dendrons are cleaved from the CPG beads using 30% Ammonium Hydroxide overnight or a 1:1 mixture of 30% Ammonium Hydroxide and Methylamine for 30 minutes. DNA dendrons are then purified by either HPLC or denaturing PAGE. Purified product is characterized by MALDI-TOF MS and PAGE.

### Example 2

### Synthesis and Testing of Oligonucleotide Dendrons

Before studying how oligonucleotide dendrons could be used to impart SNA properties on any nanoparticle core, it was necessary to first demonstrate that the oligonucleotide could elicit SNA properties on their own. This example shows that, indeed, oligonucleotide dendrons significantly outperform linear oligonucleotides in achieving cellular uptake across multiple cell lines, with the most pronounced difference being in antigen presenting immune cells. Furthermore, these initial experiments allowed for the investigation of the impact of valency, or the number of branches on the dendron, as well as the impact of dendron design (e.g., length, flexibility, size, etc.) on cellular uptake. These results confirmed that a cluster of DNA can elicit SNA properties and informed the design for future oligonucleotide dendrons.

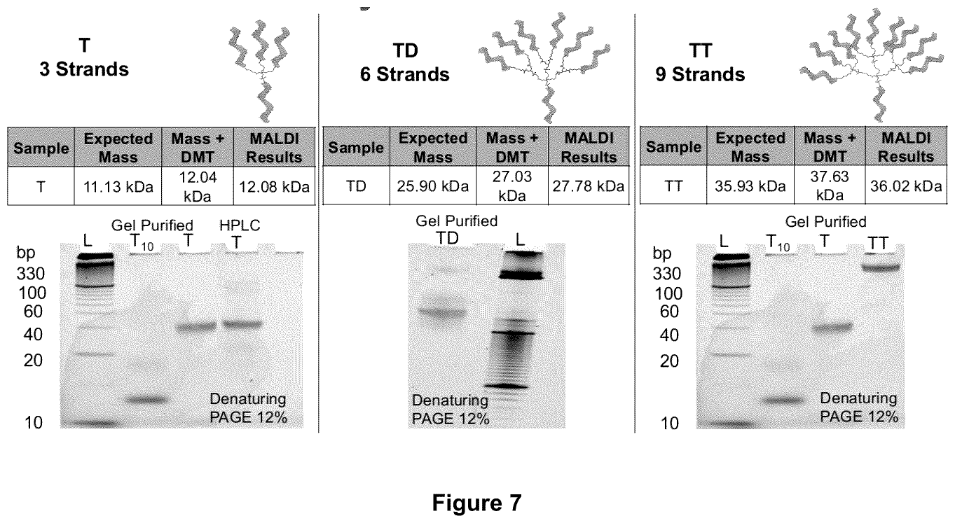

DNA dendrons were synthesized using an automated ABI DNA synthesizer on 2000 angstrom controlled pore glass (CPG) beads, commonly used in solid-phase DNA synthesis. Phosphoramidite branching units (e.g., doubler moieties, trebler moieties, or a combination thereof) were purchased from Glen Research and applied to this synthesis to access a dendritic DNA architecture (FIG. 1A). Optimal synthetic conditions were determined by systematically changing DNA length, sequence, flexibility, and branching unit design. The resultant molecules are purified by 8-15% denaturing polyacrylamide gel electrophoresis (PAGE). Purified products are characterized by Time of Flight Matrix-Assisted Laser Desorption/Ionization Mass Spectrometry (MALDI-TOF) (FIG. 1B) and PAGE (FIG. 1C). See also FIG. 7.

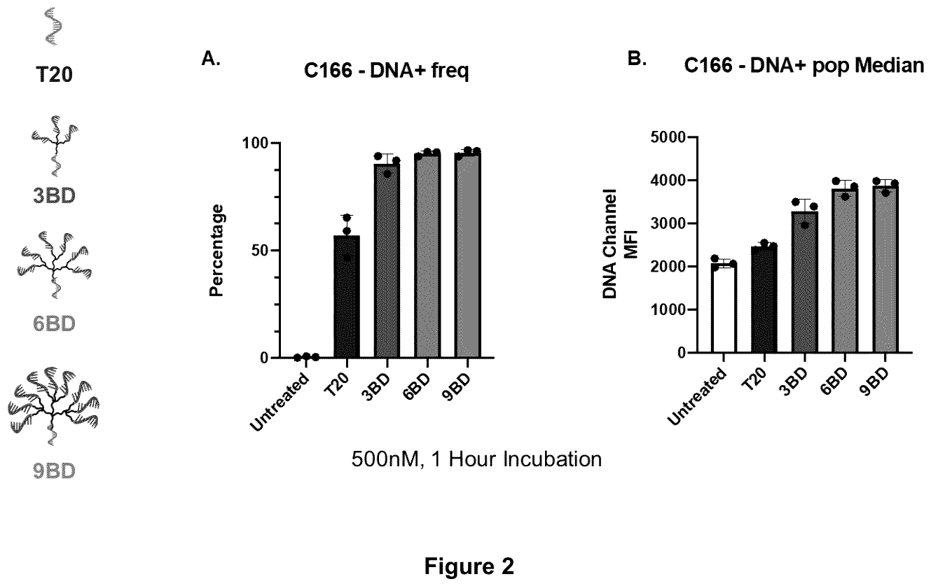

Cellular uptake of DNA dendrons with increasing valency was tested in two different cell lines. Uptake was measured using flow cytometry. When treated at 500 nM for 1 hour, C166 endothelial cells showed a significant increase in cellular uptake for all dendrons when compared to a linear control. A significant increase in both the percent of cells positive for DNA (FIG. 2A) and in the amount of DNA in each cell (FIG. 2B) was observed.

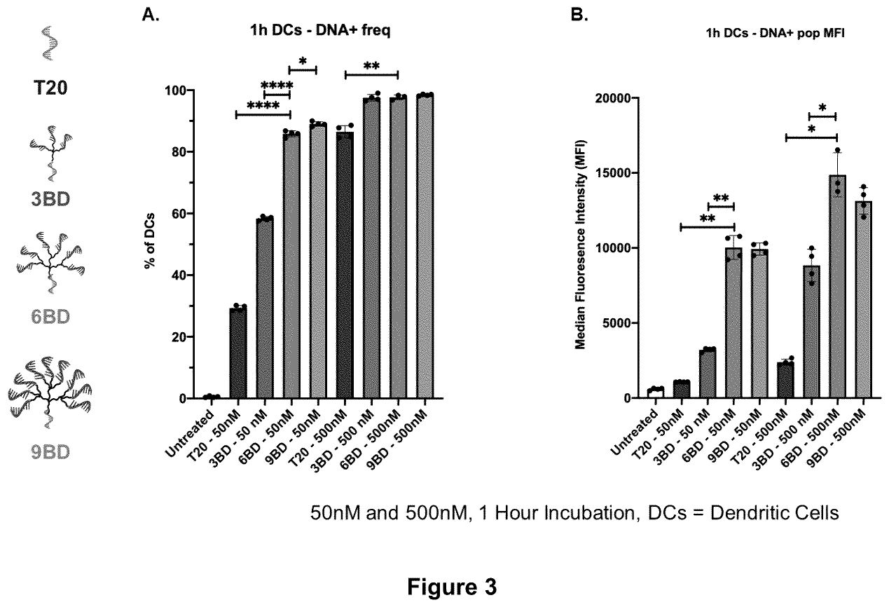

When tested in antigen presenting cells (APCs), the cells that make up the immune system, the difference in cellular uptake became more pronounced. It was observed that the six and nine branched DNA dendrons far outcompeted the three branched and linear DNA control, across a large range of concentrations (FIGS. 3A and 3B).

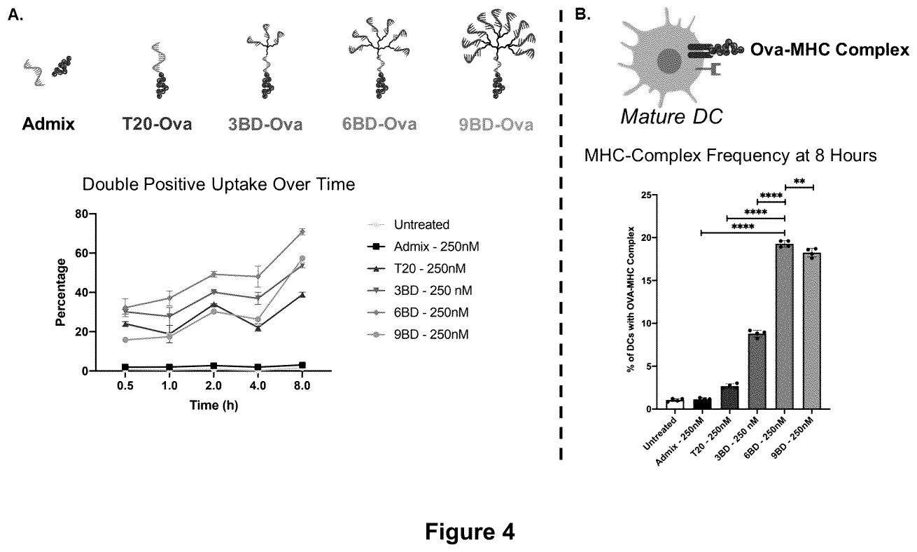

Use of DNA dendrons as a tag for intracellular delivery was tested by first conjugating the dendrons to a fluorescently tagged model peptide, Ovalbumin 1 peptide (Oval). Oval was received, containing a single cysteine residue located at the N-terminus of the peptide. The oligonucleotide dendrons were synthesized to contain an amino functional group on the 3′ end of the oligonucleotide stem. To the amino group on the dendron, the crosslinker succinimidyl 3-(2-pyridyldithio)propionate was conjugated by reacting the amino group with the NHS-ester end of the crosslinker. The dendron crosslinker conjugates were purified and reacted with the cysteine group on the Oval peptide through a disulfide exchange reaction. The resultant peptide-dendron conjugates were purified by denaturing PAGE and characterized by MALDI-TOF MS. Uptake was measured using flow cytometry to measure the percent of cells that contained both peptide and dendron. Over time, it was observed that the six branched dendron-Ova conjugate was taken up significantly more than the other samples (FIG. 4A). Thus, the six branched dendron performed better than the nine branched dendron. Moreover, these results demonstrated that attachment of a single dendron is sufficient to elicit SNA properties on materials that were previously ruled out as potential SNA cores. Furthermore, it was also observed that the delivered peptide remained functional and could bind to its specific receptor on the cell surface (FIG. 4B).

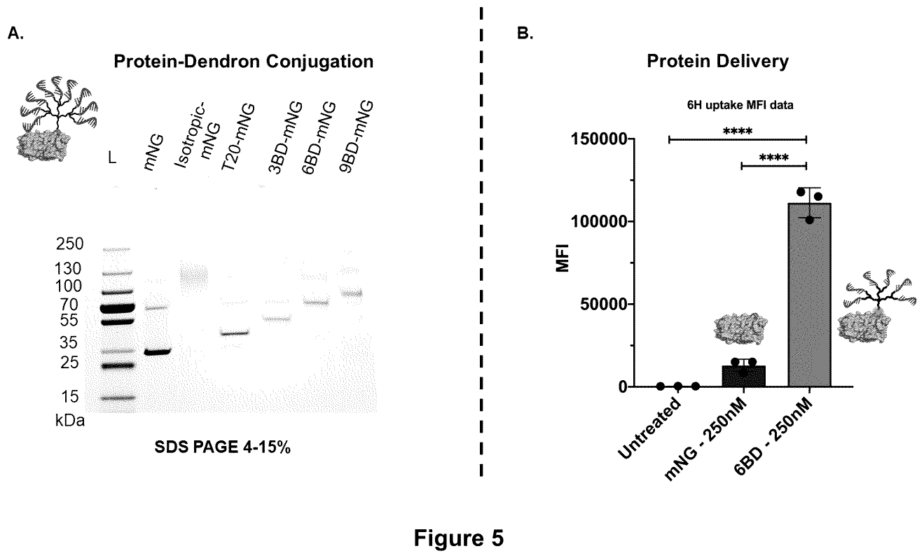

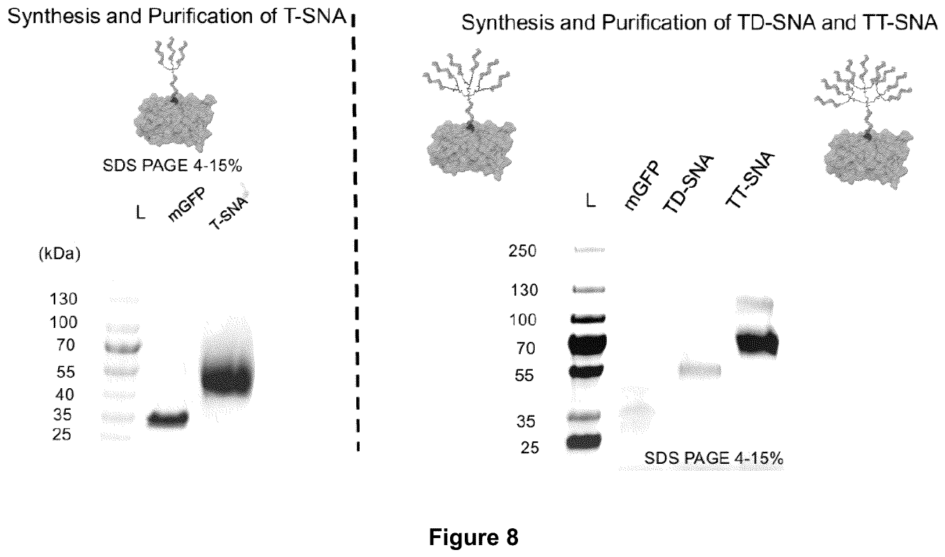

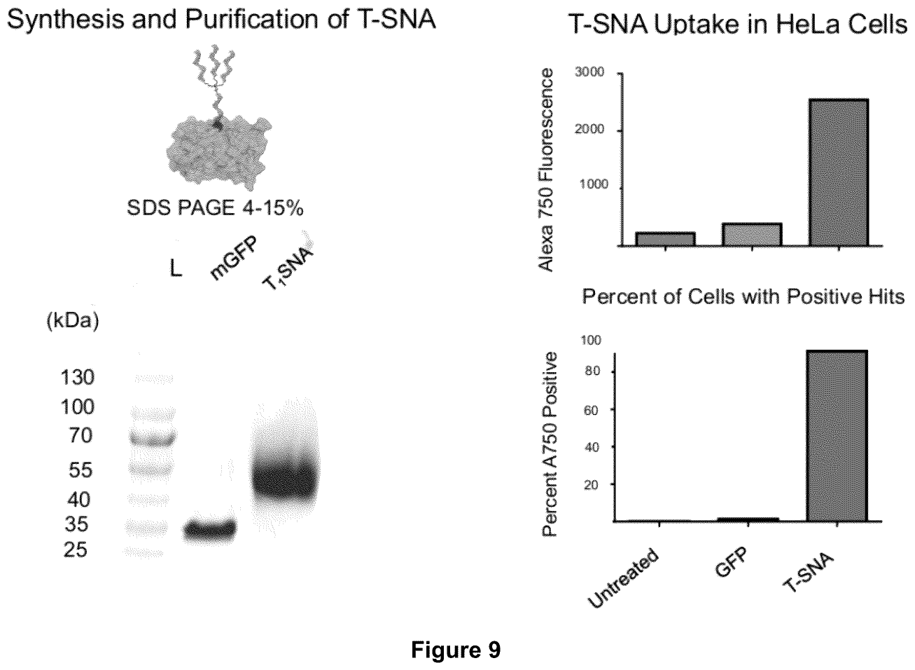

The DNA dendrons were conjugated to proteins to test whether the foregoing properties translated to larger cargo. A model fluorescent protein, mNeonGreen (mNG), was used, whereby a single DNA dendron was conjugated to its surface (FIG. 5A). The protein, mNG, was mutated to contain a single surface cysteine residue. The oligonucleotide dendrons were synthesized to contain an amino functional group on the 3′ end of the oligonucleotide stem. To the amino group on the dendron, the crosslinker succinimidyl 3-(2-pyridyldithio)propionate was conjugated by reacting the amino group with the NHS-ester end of the crosslinker. The dendron crosslinker conjugates were purified and reacted with the cysteine group on the surface of mNG through a disulfide exchange reaction. The resultant mNG-dendron conjugates were purified by denaturing PAGE and characterized by MALDI-TOF MS, UV-vis, and PAGE. Similarly, FIG. 8 shows the synthesis of additional protein-oligonucleotide dendron SNAs. It was found that, after treating cells for 6 hours, significantly more mNeonGreen is taken up when conjugated to the six branched dendron over the naked protein (FIG. 5B). See also FIG. 9, which shows cellular uptake of another protein-oligonucleotide dendrimer SNA. The results shown in FIGS. 5 and 9 demonstrate that effective cellular uptake of a protein was achieved when the protein has only a single oligonucleotide dendron attached thereto.

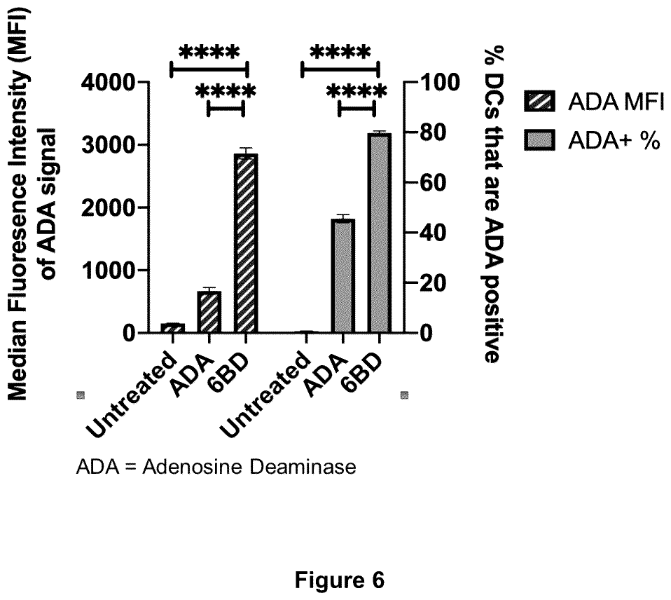

Finally, this approach was applied to a functional immunogenic protein, adenosine deaminase (ADA), which is currently used clinically to treat Severe Combined Immunodeficiency (SCID). ADA dendron conjugates were prepared by first reacting the several surface lysine residues on ADA with the crosslinker, NHS Ester-PEG4-Azide, whereby the NHS ester reacted with the lysine residues. Protein-crosslinker conjugates were purified using size exclusion chromatography. Oligonucleotide dendrons were synthesized to contain a DBCO functional group on the 3′ end of the oligonucleotide stem. The DBCO terminated oligonucleotide dendrons were reacted with the protein-crosslinker conjugates though the DBCO-azide copper free click reaction. Protein-dendron conjugates were purified by size exclusion chromatography and characterized by UV-vis, MALDI-TOF MS, and PAGE. It was shown that by conjugating DNA dendrons to the surface of ADA, significantly greater cellular uptake compared to that of the naked protein was achieved (FIG. 6).

All references, including publications, patent applications, and patents, cited herein are hereby incorporated by reference to the same extent as if each reference were individually and specifically indicated to be incorporated by reference and were set forth in its entirety herein.

What is claimed is:

1. An oligonucleotide dendron comprising an oligonucleotide stem linked to a plurality of oligonucleotide branches through one or more of a doubler moiety, a trebler moiety, or a combination thereof, the oligonucleotide stem comprises: (i) an inhibitory oligonucleotide (ii) an immunostimulatory oligonucleotide; or (iii) a toll-like receptor (TLR) antagonist.

2. The oligonucleotide dendron of claim 1, wherein each oligonucleotide branch in the plurality of oligonucleotide branches is single-stranded.

3. The oligonucleotide dendron of claim 1, wherein the oligonucleotide stem comprises an inhibitory oligonucleotide.

4. The oligonucleotide dendron of claim 1, wherein the oligonucleotide stem comprises an immunostimulatory oligonucleotide.

5. The oligonucleotide dendron of claim 4, wherein the immunostimulatory oligonucleotide comprises a toll-like receptor (TLR) agonist.

6. The oligonucleotide dendron of claim 1, wherein the oligonucleotide stem comprises a TLR antagonist.

7. The oligonucleotide dendron of claim 1, wherein the oligonucleotide stem comprises at least one phosphorothioate linkage or at least one phosphorodithioate linkage.

8. The oligonucleotide dendron of claim 1, wherein the oligonucleotide stem is about 5 to about 20 nucleotides in length.

9. The oligonucleotide dendron of claim 1, wherein the oligonucleotide stem is single-stranded.

10. The oligonucleotide dendron of claim 1, wherein the oligonucleotide stem is double-stranded.

11. The oligonucleotide dendron of claim 2, wherein the oligonucleotide stem is single-stranded.

12. The oligonucleotide dendron of claim 1, wherein the oligonucleotide dendron is a DNA dendron, a RNA dendron, a modified oligonucleotide dendron, or a combination thereof.

13. The oligonucleotide dendron of claim 3, wherein the oligonucleotide dendron is a DNA dendron.

14. The oligonucleotide dendron of claim 1, wherein the oligonucleotide dendron comprises about 2 to about 27 oligonucleotide branches.

15. The oligonucleotide dendron of claim 1, wherein the oligonucleotide dendron comprises 6 oligonucleotide branches, or the oligonucleotide dendron comprises 9 oligonucleotide branches.

16. The oligonucleotide dendron of claim 3, wherein the inhibitory oligonucleotide is antisense DNA, small interfering RNA (siRNA), an aptamer, a short hairpin RNA (shRNA), a DNAzyme, or an aptazyme.

17. The oligonucleotide dendron of claim 1, wherein the oligonucleotide stem comprises a peptide or a small molecule attached thereto.

18. The oligonucleotide dendron of claim 1, wherein the oligonucleotide stem comprises an antigenic peptide attached thereto.

19. The oligonucleotide dendron of claim 1, wherein the oligonucleotide stem comprises a peptide attached thereto, and the peptide remains functional following delivery of the oligonucleotide dendron to a cell.

20. The oligonucleotide dendron of claim 1, wherein one or more of the plurality of oligonucleotide branches comprises a peptide or a small molecule attached thereto.

21. The oligonucleotide dendron of claim 1, wherein one or more of the plurality of oligonucleotide branches comprises an antigenic peptide attached thereto.

22. The oligonucleotide dendron of claim 1, wherein one or more of the plurality of oligonucleotide branches comprises a peptide attached thereto, and the peptide remains functional following delivery of the oligonucleotide dendron to a cell.

23. A method of inhibiting expression of a gene product comprising the step of hybridizing a polynucleotide encoding the gene product with the oligonucleotide dendron of claim 2, wherein hybridizing between the polynucleotide and the oligonucleotide stem of the oligonucleotide dendron occurs over a length of the polynucleotide with a degree of complementarity sufficient to inhibit expression of the gene product.

24. The method of claim 23 wherein expression of the gene product is inhibited in vivo.

25. The method of claim 23 wherein expression of the gene product is inhibited in vitro.

26. A method for up-regulating activity of a toll-like receptor (TLR), comprising contacting a cell having the toll-like receptor with the oligonucleotide dendron of claim 5.

27. The method of claim 26, wherein the toll-like receptor is chosen from the group consisting of toll-like receptor 1, toll-like receptor 2, toll-like receptor 3, toll-like receptor 4, toll-like receptor 5, toll-like receptor 6, toll-like receptor 7, toll-like receptor 8, toll-like receptor 9, toll-like receptor 10, toll-like receptor 11, toll-like receptor 12, and toll-like receptor 13.

28. The method of claim 26 which is performed in vivo.

29. A method for down-regulating activity of a toll-like receptor (TLR), comprising contacting a cell having the toll-like receptor with the oligonucleotide dendron of claim 6.

30. The method of claim 29 which is performed in vivo.
